SEQUENCE LISTING (22 sequences)

SEQ ID NO: 1 - DNA, 20 bp, synthetic construct
  misc_feature 1..20: note = synthetic oligonucleotide
  source 1..20: mol_type = other DNA
ccagcacgatgaagatcaag

SEQ ID NO: 2 - DNA, 20 bp, synthetic construct
  misc_feature 1..20: note = synthetic oligonucleotide
  source 1..20: mol_type = other DNA
gtggacaatgaggccagaat

SEQ ID NO: 3 - DNA, 23 bp, synthetic construct
  misc_feature 1..23: note = synthetic oligonucleotide
  source 1..23: mol_type = other DNA
tctttcagcaaggactggtcttt

SEQ ID NO: 4 - DNA, 22 bp, synthetic construct
  misc_feature 1..22: note = synthetic oligonucleotide
  source 1..22: mol_type = other DNA
catccacaccattgggagtaaa

SEQ ID NO: 5 - DNA, 20 bp, synthetic construct
  misc_feature 1..20: note = synthetic oligonucleotide
  source 1..20: mol_type = other DNA
attccagctgtgagaatggg

SEQ ID NO: 6 - DNA, 20 bp, synthetic construct
  misc_feature 1..20: note = synthetic oligonucleotide
  source 1..20: mol_type = other DNA
cggtcacacttgtagggttt

SEQ ID NO: 7 - DNA, 18 bp, synthetic construct
  misc_feature 1..18: note = synthetic oligonucleotide
  source 1..18: mol_type = other DNA
acgcgtgtgtgacaatga

SEQ ID NO: 8 - DNA, 19 bp, synthetic construct
  misc_feature 1..19: note = synthetic oligonucleotide
  source 1..19: mol_type = other DNA
cactatgctgtggctggaa

SEQ ID NO: 9 - DNA, 22 bp, synthetic construct
  misc_feature 1..22: note = synthetic oligonucleotide
  source 1..22: mol_type = other DNA
aggagcttcttgtgtggtattg

SEQ ID NO: 10 - DNA, 22 bp, synthetic construct
  misc_feature 1..22: note = synthetic oligonucleotide
  source 1..22: mol_type = other DNA
cttaggatggctctgtgtttgt

SEQ ID NO: 11 - DNA, 21 bp, synthetic construct
  misc_feature 1..21: note = synthetic oligonucleotide
  source 1..21: mol_type = other DNA
cccatagagccaagcataagg

SEQ ID NO: 12 - DNA, 20 bp, synthetic construct
  misc_feature 1..20: note = synthetic oligonucleotide
  source 1..20: mol_type = other DNA
ttgacgtggtcagctctttc

SEQ ID NO: 13 - DNA, 21 bp, synthetic construct
  misc_feature 1..21: note = synthetic oligonucleotide
  source 1..21: mol_type = other DNA
ctggaacagcaagtggtagat

SEQ ID NO: 14 - DNA, 20 bp, synthetic construct
  misc_feature 1..20: note = synthetic oligonucleotide
  source 1..20: mol_type = other DNA
caggcgctgtcttaactctt

SEQ ID NO: 15 - DNA, 20 bp, synthetic construct
  misc_feature 1..20: note = synthetic oligonucleotide
  source 1..20: mol_type = other DNA
agtccctgctgcaactttag

SEQ ID NO: 16 - DNA, 21 bp, synthetic construct
  misc_feature 1..21: note = synthetic oligonucleotide
  source 1..21: mol_type = other DNA
gggatcagtcattcgctttct

SEQ ID NO: 17 - DNA, 19 bp, synthetic construct
  misc_feature 1..19: note = synthetic oligonucleotide
  source 1..19: mol_type = other DNA
tcctggacaggaaggaagt

SEQ ID NO: 18 - DNA, 21 bp, synthetic construct
  misc_feature 1..21: note = synthetic oligonucleotide
  source 1..21: mol_type = other DNA
gagtagctgccctcatctttc

SEQ ID NO: 19 - DNA, 19 bp, synthetic construct
  misc_feature 1..19: note = synthetic oligonucleotide
  source 1..19: mol_type = other DNA
ccatcatctgctgcttcct

SEQ ID NO: 20 - DNA, 20 bp, synthetic construct
  misc_feature 1..20: note = synthetic oligonucleotide
  source 1..20: mol_type = other DNA
gcttccatccagtgatcctt

SEQ ID NO: 21 - DNA, 21 bp, synthetic construct
  misc_feature 1..21: note = synthetic oligonucleotide
  source 1..21: mol_type = other DNA
gtctccaagcaagccccatta

SEQ ID NO: 22 - DNA, 32 bp, synthetic construct
  misc_feature 1..32: note = synthetic oligonucleotide
  source 1..32: mol_type = other DNA
tcgcaagctttaccagggtctttggacacctc

(19) United States
(12) Patent Application Publication
(10) Pub. No.: US 2024/0402173 A1
(43) Pub. Date: Dec. 5, 2024

(54) IgE+ PLASMABLASTS AS A PREDICTIVE BIOMARKER OF ALLERGY

(71) Applicant: CORNELL UNIVERSITY, Ithaca, NY (US)

(72) Inventors: Bettina WAGNER, Ithaca, NY (US); Elisabeth SIMONIN, Brooktondale, NY (US); Susanna BABASYAN, Ithaca, NY (US)

(73) Assignee: CORNELL UNIVERSITY, Ithaca, NY (US)

(21) Appl. No.: 18/699,663

(22) PCT Filed: Oct. 14, 2022
(86) PCT No.: PCT/US2022/078106

§ 371 Date: Apr. 9, 2024

Related U.S. Application Data
(60) Provisional application No. 63/255,756, filed on Oct. 14, 2021.

(51) Int. Cl. G01N 33/569; G01N 33/577; G01N 33/68

(52) U.S. Cl. CPC G01N 33/56972; G01N 33/577; G01N 33/6854; G01N 2333/70596

(57) ABSTRACT

Methods of detecting IgE+ plasmablasts in a mammal are described herein. Also described herein are methods of predicting clinical allergy in a mammal. Additionally, methods of monitoring progression of an allergy along with methods of monitoring efficacy of treatment of an allergy are described.

### CROSS REFERENCE TO RELATED APPLICATIONS

This application claims the benefit of U.S. Provisional Patent Application No. 63/255,756, filed Oct. 14, 2021, the contents of which is incorporated herein by reference in its entirety.

### BACKGROUND

IgE-mediated allergic diseases arise when allergen-specific B cells class switch to produce IgE, which then binds to effector mast cells and basophils to promote clinical allergy (doi: 10.1038/nri2273). B cells differentiate into antibody secreting peripheral plasmablasts before becoming plasma cells (doi: 10.3389/fimmu.2012.00078; doi: 10.1016/B978-0-12-397933-9.00014-X). Recent single-cell RNA sequencing suggested that the majority of human peripheral IgE+ B cells may actually be differentiating plasmablasts (doi: 10.1126/science.aau2599).

While well accepted in allergy biology, the study of IgE+ B cells and plasmablasts prove difficult in large part due to the expression of CD23, the low affinity IgE receptor, and binding of IgE by CD23 on the cell surface of most activated B cells. Typically, identification of IgE+ B cells has required either exclusion of all other B cell classes, where IgM−/IgD−/IgG−/IgA− B cells are assumed to be IgE+ (doi: 10.1016/j.jaci.2014.03.036; doi: 10.1111/all.13421; and doi: 10.1016/j.jaci.2019.04.001), or intracellular staining of IgE production (doi: 10.1111/all.12679), which prevents live cell functional analysis. The quantification of IgE-expressing B cells in these studies likely overestimated their frequency (doi: 10.1016/j.jaci.2019.04.001). An anti-human IgE monoclonal antibody that can distinguish between membrane expressed and receptor-bound IgE has also been used to label IgE+ human B cells (doi: 10.3390/cells8090994).

IgE+ B cells measured in these ways are increased in humans with atopic dermatitis and food allergy, compared to healthy controls (doi: 10.1016/j.jaci.2014.03.036; doi: 10.1111/all.13421), and may also increase following allergen exposure (doi: 10.3390/cells8090994). However, due to the difficulty of characterizing these cells for analysis, the relationship between allergen exposure and the presence of IgE+ B cells in peripheral blood is incompletely understood. Likewise, rare peripheral IgE+ plasmablasts and plasma cells have been identified but their relationship to clinical allergy is less defined (doi: 10.1126/science.aau2599; doi: 10.1046/j.1365-2249.2002.02025.x). Most prior studies do not differentiate between IgE+ B cells and IgE+ plasmablasts.

Immunoglobulin E (IgE) is the antibody that causes allergic reactions through binding of effector cells and stimulation by specific allergens (doi: 10.1038/nm.2755). Allergen-specific IgE is often used to diagnose allergy development and severity (doi: 10.1586/1744666X.2014.872032; doi: 10.1007/si1882-018-0816-4). Allergen-specific serum IgE has also been used to guide which allergens should be included in allergen immunotherapy (doi: 10.1016/j.anai.2013.07.005) and to predict treatment effectiveness (doi: 10.1007/s11882-018-0816-4). However, the concentration of allergen-specific IgE in circulation can be low and the identification of causal allergens is difficult due in part to cross reactivity between major and minor allergens (doi: 10.1111/cea.13432). Additionally, total IgE levels may not predict treatment success, for example as was seen in an Omalizumab trial (doi: 10.1016/j.rmed.2007.01.011). Due to this variability, allergic patients can be surprised by unexpected allergic reactions to new allergens or left unsure of the true disease severity.

IgE is produced when allergen-specific B cells are activated by allergen in the presence of the cytokines IL-4 and IL-13 (doi: 10.1084/jem.168.3.853; doi: 10.1073/pnas.90.8.3730), leading to class switch recombination of the constant region to express the IGHE gene (doi: 10.1016/s0165-2427(01)00355-5; doi: 10.1016/j.jaci.2015.07.014). These class-switched B cells, now expressing an IgE B cell receptor, then receive additional survival signals and begin differentiating into memory B cells and plasma cells, the latter secrete high concentrations of IgE into circulation (doi: 10.1016/j.immuni.2006.12.006; doi: 10.7554/eLife.21238; doi: 10.1126/science.aau2599). During the activation process, some IgE+ B cells differentiate into IgE+ plasmablasts. IgE+ plasmablasts secrete antibody and enter peripheral blood, providing a snapshot of the IgE+ B cell to plasma cell differentiation process simultaneously occurring in the lymph node or local tissue. Peripheral IgE+ plasmablasts may continue to the bone marrow where they further differentiate into plasma cells (doi: 10.1038/nri3795).

Different mammalian species can experience naturally occurring IgE-mediated allergic diseases, including humans, horses, dogs, and cats. The horse has been used as a model for human allergy due to the many similarities in the immune mechanism of disease (doi: 10.1016/j.vetimm.2012.02.007; doi: 10.1016/j.molimm.2021.04.013). Horses also allow the ability to control key variables, such as uniform environment and allergen exposure, identical living conditions for allergic and control animals, and identical treatment for allergic animals, if any. Horses express CD23 on peripheral B cells, and previous work has identified that most equine CD23+ B cells are either IgM+ or IgG+ B cells (doi: 10.1016/j.vetimm.2012.02.007).

High CD23 expression on IgE+ plasmablasts has been first identified by studies in horses. Recent single cell sequencing of human B cell surface proteins identified that some human individuals have two distinct B cell populations based on CD23 expression (CD23^{lo} and CD23^{hi}), suggesting that CD23^{hi} IgE+ plasmablasts also exist in humans (doi: 10.1038/s42003-020-1075-1). This also supports the thought that heterogeneity in CD23 function on B cells and plasmablasts is conserved amongst mammals, the only species which produce IgE. However, the cause and clinical relevance of these two populations were not explored in humans. Similarly, another recent study noted that allergic people have CD23^{hi} class switched B cells that are absent in healthy controls (doi: 10.1111/all.14288).

No additional studies identify or measure IgE+ plasmablasts in species prone to allergy. Likewise, there are no biomarkers to determine whether allergen immunotherapy treatment is effectively desensitizing the individual.

### BRIEF SUMMARY OF THE DISCLOSURE

The present disclosure is directed to a method of detecting IgE+ plasmablasts in a mammal, a method of predicting clinical allergy in a mammal, a method of determining the severity of clinical allergy in a mammal, a method for monitoring progression of an allergy in a mammal, a method for monitoring the efficacy of an allergy treatment regimen in a mammal, and a kit for the use of detecting IgE+ plasmablasts in a mammal.

In a first aspect, the present disclosure is directed to a method of detecting IgE+ plasmablasts in a mammal, the method comprising: (i) contacting peripheral blood leukocytes (PBL) or peripheral blood mononuclear cells (PBMC) isolated from a blood sample of the mammal, with a wash solution, followed by contacting the washed PBL or PBMC with an acidic wash solution, and thereafter collecting the cells; then (ii) contacting the collected cells from step (i) with a detection agent directed to IgE; and (iii) detecting IgE+ cells as IgE+ plasmablasts. In some embodiments, the PBL or PBMC are first washed with a wash solution at a pH range of 4-6. In some embodiments, steps (ii) and (iii) comprise (ii) contacting the collected cells from step (i) with a detection agent directed to CD23 and a detection agent directed to IgE; and (iii) detecting CD23+ and IgE+ cells as IgE+ plasmablasts. In some embodiments, the acidic wash solution has a pH in the range of 2.5-3.1. In some embodiments, the acidic wash solution has a pH in the range of 2.8-3.0. In some embodiments, the acidic wash solution has a pH of about 3.0. In some embodiments, the cells collected from step (i) are contacted with the acidic wash solution for about 3-10 minutes. In some embodiments, the acidic wash solution is a lactic acid solution. In some embodiments, the pH is neutralized before the cells are collected. In some embodiments, the detection agent directed to CD23 is a monoclonal antibody, and the detection agent directed to IgE is a monoclonal antibody. In some embodiments, the detection is achieved by a flow cytometer. In some embodiments, the PBLs or PBMCs are obtained by removing red blood cells from a blood sample of the mammal. In some embodiments, the mammal is a human, a horse, a dog, or a cat. In some embodiments, the method of detecting IgE+ plasmablasts in a mammal, further includes determining the percentage of IgE+ cells in the total CD23+ cells.

In some embodiments, the disclosure is directed to a method of quantifying IgE, the method comprising:
culturing peripheral blood leukocytes (PBL) or peripheral blood mononuclear cells (PBMC) isolated from a blood sample of the mammal,
measuring IgE secreted from the cultured cells in the supernatant; and quantifying the level of secreted IgE.

In some embodiments, the culturing is performed for a period of about 6 hours to about 96 hours. In some embodiments, the measuring is achieved by a laminar flow device, ELISA, bead-based assay, or similar assay platforms. In some embodiments, the mammal is a human, a horse, a dog, or a cat. In some embodiments, the level of secreted IgE reflects the percentage of CD23+ and IgE+ cells in the total CD23+ cells.

Another aspect of the disclosure is directed to a method of predicting clinical allergy in a mammal, the method comprising detecting IgE+ plasmablasts in the mammal, and determining the risk of developing clinical allergy based on the level of IgE+ plasmablasts. In some embodiments, the IgE+ plasmablasts are detected using the methods disclosed herein. In some embodiments, the level of IgE+ plasmablasts is determined based on the percentage of CD23+ and IgE+ cells in the total CD23+ cells. In some embodiments, the mammal will likely develop clinical allergy if the percentage of CD23+ and IgE+ cells in the total CD23+ cells is above a range of 10% to 14.%. In some embodiments, the level of IgE+ plasmablasts is determined based on the percentage of IgE+ cells in the total sample. In some embodiments, the mammal will likely develop clinical allergy if the percentage of IgE+ cells in total cells is above a range of 0.1-0.15%. In some embodiments, the method further comprises treating the mammal with antihistamines, allergen immunotherapy, or biologics (i.e. monoclonal antibodies) aimed to desensitize the individual to allergens when it is determined that the mammal will likely develop clinical allergy. In some embodiments, the method further comprises treating the mammal by dialyzing the IgE+ CD23+ cells isolated from the mammal. In some embodiments, the mammal is a human, a horse, a dog, or a cat.

Another aspect of the disclosure is directed to a method of predicting clinical allergy in a mammal, the method comprising detecting secreted IgE in the mammal, and determining the risk of developing clinical allergy based on the level of secreted IgE. In some embodiments, the secreted IgE is detected using the methods disclosed herein. In some embodiments, the mammal will likely develop clinical allergy if the level of secreted IgE is above a predetermined threshold level. In some embodiments, the method further comprises treating the mammal with antihistamines, allergen immunotherapy, or biologics (i.e. monoclonal antibodies) aimed to desensitize the individual to allergens when it is determined that the mammal will likely develop clinical allergy. In some embodiments, the mammal is a human, a horse, a dog, or a cat.

In another aspect, the disclosure is directed to a method of determining the severity of clinical allergy in a mammal, comprising detecting IgE+ plasmablasts in the mammal, and determining the severity of clinical allergy based on the level of IgE+ plasmablasts. In some embodiments, the IgE+ plasmablasts are detected using the methods disclosed herein. In some embodiments, the level of IgE+ plasmablasts is determined based on the percentage of CD23+ and IgE+ cells in the total CD23+ cells. In some embodiments, the mammal will have more severe allergy based on an increase in the percentage of CD23+ and IgE+ cells in the total CD23+ cells above a range of 10% to 14.%. In some embodiments, the level of IgE+ plasmablasts is determined based on the percentage of IgE+ cells in the total sample. In some embodiments, the mammal will likely develop more severe allergy based on an increase in the percentage of IgE+ cells in total cells above a range of 0.1-0.15%. In some embodiments, the method further comprises treating the mammal with antihistamines, allergen immunotherapy, or biologics aimed to desensitize the individual to allergens when it is determined that the mammal will likely develop clinical allergy. In some embodiments, the method further comprises treating the mammal by dialyzing the IgE+ CD23+ cells isolated from the mammal. In some embodiments, the mammal is a human, a horse, a dog, or a cat.

Another aspect of the disclosure is directed to a method of determining the severity of clinical allergy in a mammal, comprising detecting secreted IgE in the mammal, and determining the severity of clinical allergy based on the level of secreted IgE. In some embodiments, the secreted IgE is detected using the methods disclosed herein. In some embodiments, the mammal will have more severe allergy based on an increase in the level of secreted IgE that surpasses a predetermined threshold. In some embodiments, the method further comprises treating the mammal with antihistamines, allergen immunotherapy, or biologics aimed to desensitize the individual to allergens when it is determined that the mammal will likely develop clinical allergy. In some embodiments, the mammal is a human, a horse, a dog, or a cat.

Another aspect of the disclosure is directed to a method for monitoring progression of an allergy comprising monitoring the level of IgE+ plasmablasts in a mammal, and determining that allergy is progressing based on an increase in the level of IgE+ plasmablasts. In some embodiments, the IgE+ plasmablasts are detected using methods disclosed herein. In some embodiments, the level of IgE+ plasmablasts is determined based on the percentage of CD23+ and IgE+ cells in the total CD23+ cells. In some embodiments, the level of IgE+ plasmablasts is determined based on the percentage of IgE+ cells in the total sample. In some embodiments, the mammal is a human, a horse, a dog, or a cat.

Another aspect of the disclosure is directed to a method for monitoring progression of an allergy comprising monitoring the level of secreted IgE in a mammal, and determining that allergy is progressing based on an increase in the level of secreted IgE. In some embodiments, the secreted IgE is detected using methods disclosed herein. In some embodiments, the mammal is a human, a horse, a dog, or a cat.

One aspect of the disclosure is directed to a method for monitoring the efficacy of a treatment regimen for allergy, comprising monitoring the level of IgE+ plasmablasts in a mammal undergoing the treatment regimen for allergy, and determining the efficacy of the treatment regimen based on the level of IgE+ plasmablasts, wherein a decrease in the level of IgE+ plasmablasts indicates that the treatment regimen is effective. In some embodiments, the IgE+ plasmablasts are detected using methods disclosed herein. In some embodiments, the level of IgE+ plasmablasts is determined based on the percentage of CD23+ and IgE+ cells in the total CD23+ cells. In some embodiments, the level of IgE+ plasmablasts is determined based on the percentage of IgE+ cells in the total sample. In some embodiments, the mammal is a human, a horse, a dog, or a cat.

Another aspect of the disclosure is directed to a method for monitoring the efficacy of a treatment regimen for allergy, comprising monitoring the level of secreted IgE in a mammal undergoing the treatment regimen for allergy, and determining the efficacy of the treatment regimen based on the level of secreted IgE, wherein a decrease in the level of secreted IgE indicates that the treatment regimen is effective. In some embodiments, the secreted IgE is detected using methods disclosed herein. In some embodiments, the mammal is a human, a horse, a dog, or a cat.

Another aspect of the disclosure is directed to a kit for detecting IgE+ plasmablasts, comprising an acidic wash solution, a monoclonal anti-CD23 antibody, and a monoclonal anti-IgE antibody. In some embodiments, the kit further comprises instructions for use. In some embodiments, the anti-CD23 antibody and the anti-IgE antibody are of different species. In some embodiments, the anti-CD23 antibody is against horse CD23 and the anti-IgE antibody is against horse IgE. In some embodiments, the anti-CD23 antibody is against human CD23 and the anti-IgE antibody is against human IgE. In some embodiments, the kit further comprises labeled detection antibodies against the anti-CD23 antibody and the anti-IgE antibody monoclonal. In some embodiments, the monoclonal anti-CD23 antibody and the monoclonal anti-IgE antibody are coupled to different fluorescent dyes.

### BRIEF DESCRIPTION OF THE DRAWINGS

FIG. 1A-D. IgE⁺ plasmablasts appear in peripheral blood before the onset of clinical allergy. PBL were frequently collected from heparinized blood samples for the duration of one year to compare the onset of IgE+ plasmablasts in peripheral blood and the development of clinical allergy. Samples were collected from allergic (n=7, black circles) and healthy (n=10, open circles) horses. (A) Timepoints of sample collection. Samples were collected every 3-4 days from April 12-June 10 when midges were in the environment and horses were developing clinical allergy. Before and after this period, samples were collected every 14 days until the first frost on November 2. Samples were collected once a month in February and December. (B) Daily minimum (gray squares) and maximum (black squares) temperatures (° F.) were recorded at each timepoint. (C) Clinical scores (0-10) were assigned to each horse at each timepoint using the Cornell scoring system described by Miller et al. (doi: 10.111/vde.12784). The dotted horizontal line denotes the threshold ≥3 where allergic horses have clinical allergy. The three phases of the allergen exposure season (Rising, Chronic and Resolving) are labeled in purple, red and blue boxes, respectively. (D) The frequency of IgE+ plasmablasts was recorded at each timepoint. The dotted horizontal line represents the threshold were allergic horses have ≥12% IgE+ plasmablasts. (B-D) Gray vertical bars show high temperature peaks (May 3, May 20, June 7, July 6, October 4). (C-D) Open arrows (April 26) show the first day of IgE+ plasmablasts above the threshold of 12% in allergic horses. Black arrows (May 24) show first day when clinical allergy scores above the threshold of 3 were given to allergic horses. Graphs show mean and standard deviation.

FIG. 2A-F. IgE⁺ plasmablast quantification in PBMC or PBL. PBMC or PBL were isolated from heparinized whole blood of 17 horses on the same day. Blood collection occurred in the summer when allergic horses had clinical allergy and elevated peripheral IgE⁺ plasmablast frequencies. In A-D, PBMC samples are shown on the top and PBL samples are shown on the bottom. (A) Doublet exclusion. (B) CD23 expression on doublet excluded cells. (C) IgE and CD23 expression on CD23⁺ gated cells. Percentage reports IgE⁺ plasmablasts (IgE⁺ PB) out of total CD23⁺ cells. (D) Forward (FSC) and side scatter (SSC) characteristics of IgE⁺ plasmablasts (black) in total PBMC or PBL (gray). (E) Spearman rank correlation of the IgE⁺ plasmablast frequency in PBMC (open circles) and PBL (closed circles). (F) Spearman rank correlation of secreted IgE (μg/ml) in the supernatant of PBMC (open circles) and PBL (closed circles) after 72 hours in vitro culture. In E-F, samples from the same individual are connected. FACS images are representative from 1 out of 17 horses. Correlations were calculated with samples collected on July 19.

FIG. 3A-C. Alternative analysis with only one antibody for quantification of IgE+ plasmablasts. Following the lactic acid wash, IgE+ plasmablasts can also be identified with the use of only one fluorescently-conjugated antibody, targeted against IgE. This describes the simplified gating strategy for this invention variation. (A) Flow cytometry images are gated first to exclude doublets (left), then to gate on all IgE+ cells (middle), which are solely comprised of IgE+ plasmablasts after acid wash (right). The percentage of IgE+ cells out of total singlets (middle) and percentage of IgE+ plasmblasts out of total IgE+ cells (right) are the quantitative readouts. (B) Spearman rank correlation of the frequency of IgE+ plasmablasts (IgE+ out of CD23+) and of total IgE+ cells (out of singlets) on July 19 from allergic (n=7, black circles) and healthy (n=10, open circles) horses. (C) Variation of IgE+ plasmablasts measured as described in A between allergic and healthy individuals. Graph plots mean and standard deviation. The dotted line represents the threshold were allergic horses have ≥0.15% total IgE+ cells. FACS images are representative from 1 out of 17 horses. Statistics report Sidak's multiple comparisons test.

FIG. 4A-C. Alternative quantification by measuring spontaneous secretion of IgE by IgE+ plasmablasts in culture. The IgE+ plasmablast product, secreted IgE, can also be identified by measuring their secreted IgE after culturing of the cells. Ex vivo peripheral blood leukocytes (PBL) or peripheral blood mononuclear cells (PBMC) can be incubated at 37° C. in cell culture medium with no additional stimuli. Secreted IgE can then be measured in the culture supernatant. Here, PBMC or PBL were collected from allergic (closed circles, e, black bars, n=7) and nonallergic (open circles, ∘, white bars, n=7) individuals, incubated at 37° C. with no additional stimuli, and IgE was measured in the supernatants by a Luminex bead-based assay for IgE. (A) IgE secreted from PBMC after incubation for 24, 48, 72, or 96 hours. (B) IgE can also be detected in the supernatant of PBL after 72 hours. Shorter incubation times likely still allow for quantifiable secreted IgE concentrations. (C) Spearman rank correlation of the concentration of secreted IgE from PBL after 72 hours and the frequency of IgE+ plasmablasts out of total CD23+ cells.

FIG. 5A-I. Equine CD23+ cells include a CD23^{hi}/IgM/IgG1 population. PBMC were stained for CD23 and different cell surface markers. All cells were first gated for (A) doublet exclusion and (B) cells with PBMC morphology. (C) CD23 expression on PBMC gated cells. Expression of different cell proteins were measured on CD23+ gated cells: (D) CD4 and CD8, (E) MHCII and CD14. (F) CD23+ cells can be separated into a CD23^{lo} and a CD23^{hi} population. IgM and IgG1 expression on (G) CD23^{lo} cells and (H) CD23^{hi} cells. (I) The distribution of IgM+/IgG1+ (gray, striped bars), IgM+ (gray bars), IgG1+ (white bars), and IgG1−/IgM− (black bars) expression within the CD23^{lo} and CD23^{hi} cell fractions. Graph shows the average distribution from four allergic individuals. FACS images are representative from one out of eight individuals.

FIG. 6A-F. Removal of CD23-bound IgE reveals a population that expresses IgE, and no other isotypes. PBMC were treated with a lactic acid wash solution to remove surface CD23 receptor-bound IgE. Afterwards, cells were stained for CD23 and the presence of cell surface immunoglobulin as part of their B cell receptors and measured by flow cytometry. (A) CD23 and IgE staining on ex vivo PBMC prior to the acid wash. Gates distinguish CD23^{hi} and CD23^{lo} cells. (B) CD23 and IgE expression on PBMC after treatment with a lactic acid wash solution to remove CD23 receptor-bound IgE. Gates distinguish CD23^{hi} and CD23^{lo} cells. Gated cells from B are analyzed in C-D. (C) Expression of IgE and IgM, IgD, or IgG1 on CD23^{lo} cells. (D) Expression of IgE and IgM, IgD, IgG1, IgG3/5, IgG4/7, or IgG6 on CD23^{hi} cells. (E) CD23 expression on acid washed PBMC. (F) Mouse IgG1 isotype controls on CD23+ gated cells. FACS images are representative from one out of four individuals.

FIG. 7A-F. Acid wash removes FcRI-bound IgE from monocytes and basophils and preserves IgE+ plasmablast ability to bind IgE. PBMC were treated with an acid wash solution to remove surface receptor-bound IgE. To determine if IgE was removed from FcRI, PBMC were fixed and stained to measure IgE on CD14+ monocytes and CD14− basophils. (A) CD23 expression on ex vivo PBMC prior to the acid wash. (B) CD14 and IgE staining on CD23− cells gated in A. (C) CD23 expression on acid washed PBMC. (D) CD14 and IgE staining on CD23− cells gated in C. Gates in B and D distinguish IgE-binding monocytes and basophils. FACS images are representative from one out of eight individuals. To determine if IgE+ plasmablasts could still bind IgE after acid wash, PBMC were acid washed and then incubated for 40-hours in cell culture medium. Cells were fixed and stained for surface CD23 and IgE. (E) PBMC treated with acid wash solution before incubation. Gate distinguishes IgE+ plasmablasts. (F) PBMC after 40-hour incubation in medium alone. Gate distinguishes CD23+/IgE+ cells.

FIG. 8A-K. CD23^{hi}/IgE⁺ cells produce intracellular IgE. PBMC were first treated with acid wash solution to remove CD23-bound IgE. Cells were fixed and stained for surface IgE expression, followed by intracellular IgE staining and flow cytometric analysis. (A) CD23 and cell surface IgE expression on PBMC after acid wash. Gates show CD23^{hi}/IgE⁺ cells and IgE⁻ B cells (IgE⁻ BC). (B) Mouse IgG1 isotype controls did not bind acid washed cells during extracellular incubation (A647) or intracellular incubation (A488). C-H show CD23^{hi}/IgE⁺ gated cells. (C) Surface labeling with IgE mAb-A647 was followed by (D) intracellular labeling with IgE mAb-A488 and (E) expression of both surface and intracellular IgE is shown. (F) Surface labeling with IgE mAb-A647 was followed by (G) a second surface labeling with IgE mAb-A488, and (H) labeling of both surface antibodies is shown. I-K show gated IgE⁻ BC. (I) Surface labeling with IgE mAb-A647 was followed by (J) intracellular labeling with IgE mAb-A488 and (K) expression of both surface and intracellular IgE is shown. FACS images are representative from one out of four horses.

FIG. 9A-F. Gating strategy and fluorescence activated cell sorting (FACS) outcomes for CD23hi/IgE+ cells. PBMC were isolated and acid washed to remove surface receptor-bound IgE. Afterwards, the cells were stained with mAbs for cell surface CD23 and IgE. Both, CD23hi/IgE+ cells and IgE− B cells were purified by FACS. Cell sorting purities were quantified by flow cytometry. (A-B) Gates for doublet exclusion. (C) Live cells were gated by viability dye. (D) CD23 and IgE on viable cells was used to gate on CD23hi/IgE+ cells and IgE− B-cell (IgE− BC) fractions. Purity of (E) CD23hi/IgE+ cells and (F) IgE BC in sorted fractions. Purity frequency shows mean and standard deviation of three different horses.

FIG. 10A-D. CD23^{hi}/IgE⁺ cells have upregulated plasmablast differentiation marker transcripts and exhibit plasmablast morphology. Gene expression was compared in IgE⁻ B cells (IgE⁻ BC, □, open squares, n=3) and CD23^{hi}/IgE⁺ cells (▪, closed squares, n=3). RNA was extracted, equal RNA concentrations were converted into cDNA, and cDNA was amplified by quantitative RT-PCR with gene specific primers. Data was normalized to ACTB (dCt). (A) Individual dCt values and means are graphed for the transcription factors BCL6, PAX5, BLIMP-1, IRF4 and XBP1, and surface proteins CD138, TACI and CD19. Graph plots individual values with medians. (B) Gel image showing PCR products from IGHE primers run on CD23^{hi}/IgE⁺ sorted cells. Lane 1 shows annotated ladder and lanes 2-4 show the PCR product from three different allergic individuals. (C) Sorted IgE⁻ BC and (D) sorted CD23^{hi}/IgE⁺ cells were prepared by cytospin. Perinuclear clear zone (black arrow) and cytoplasmic clear vacuoles (gray arrow) are labeled in two CD23^{hi}/IgE⁺ cells. Cells were differentially stained and imaged under 50× magnification. * p<0.05, ** p<0.01.

FIG. 11A-D. Peripheral IgE⁺ plasmablast percentages correlate with severity of clinical allergy. Percentages of peripheral blood IgE+ plasmablasts were compared in allergic (closed circles, e, black bars, n=7) and nonallergic (open circles, ∘, white bars, n=6) horses by flow cytometry. (A) Allergy scores of both horse groups during year of study. (B) CD23^{hi}/IgM⁻/IgG1⁻ cells were analyzed over one year. Percentages of CD23^{hi}/IgM⁻/IgG1⁻ cells out of total CD23+ cells are shown. (C) Percentages of IgE+ plasmablasts out of total CD23+ cells in July and December. PBMC were treated with acid wash solution before antibody staining to remove CD23-bound IgE. (D) Spearman rank correlation of the percentage of IgE+ plasmablasts in peripheral blood and allergy scores for all horses in July, during the peak of allergen exposure and clinical allergy. (A-B) The shaded boxes represent months when Cul were present in the environment. (A, D) The dotted line represents the threshold where horses with scores ≥3 have clinical allergy. (A-C) Asterisks denote months when IgE⁺ plasmablast frequency was significantly increased in allergic horses, compared to nonallergic horses. ** p<0.01 *** p<0.001.

FIG. 12A-F. IgE⁺ plasmablasts readily secrete IgE proportional to cell percentage. Functional ability to secrete IgE was compared between healthy (open circles, ∘, n=7) and allergic (closed circles, ●, n=7) horses. PBMC were incubated for 72-hours in cell culture medium and total IgE was measured in the supernatant from allergic and healthy groups. Both (A) ex vivo PBMC and (B) acid washed PBMC were incubated in parallel and IgE secretion was compared. (C) Total IgE in serum. (D) The frequency of IgE⁺ plasmablasts out of total PBMC, measured by flow cytometry after treatment with acid wash solution, before incubation. (E) Spearman rank correlation of the percentage of peripheral IgE⁺ plasmablasts and the concentration of total IgE in supernatant of ex vivo PBMC after 72-hour incubation in cell culture medium. Line shows a simple linear regression. (F) Concentration of total IgE in the supernatant of sorted IgE⁻ B cells (IgE− BC, open triangles) and IgE⁺ plasmablasts (IgE+ PB, closed triangles) from allergic horses (n=3) after 72-hour incubation in cell culture medium. Graphs plot median and individual values. * p<0.05; ** p<0.01.

FIG. 13A-F. IgE secretion, but not plasma IgE, increases before onset of clinical allergy. PBL and cell-depleted plasma samples were collected from allergic (n=7, black circles) and healthy (n=10, open circles) horses every 3-14 days for one year. The concentration of secreted IgE by PBL was evaluated in cell culture supernatants after 72 hours of in vitro culture. IgE concentrations were measured by a bead-based IgE assay. (A) Secreted IgE was compared at each timepoint. The dotted line denotes the threshold where allergic horses had ≥0.5 μg/ml secreted IgE. Black arrow shows the first day allergic horses had clinical allergy scores above the threshold of 3 (May 24). (B) Spearman rank correlation of the frequency of IgE+ plasmablasts (out of CD23+) and the concentration of secreted IgE from PBL (μg/ml). (C) Spearman rank correlation of clinical score and the concentration of secreted IgE from PBL (μg/ml). (D) Total IgE (μg/ml) in plasma was compared at each timepoint. The dotted line denotes the threshold where allergic horses had ≥100 μg/ml plasma IgE. (E) Spearman rank correlation of the frequency of IgE+ plasmablasts (out of CD23+) and the concentration of plasma IgE (μg/ml). (F) Spearman rank correlation of clinical score and the concentration of plasma IgE (μg/ml). All correlations were calculated with samples collected on July 6, during the Chronic Phase.

FIG. 14. A graphical representation of IgE+ plasmablasts comprising CD23 as well as IgE B cell receptors (BCR). IgE can bind to one of its receptors, the low-affinity IgE receptor CD23, which is expressed on activated B cells. As a result, most B cells bind IgE through CD23 on their surface. This makes the identification of IgE producing cells challenging. The disclosure provides an approach to accurately identify live IgE⁺ plasmablasts in peripheral blood for application by both flow cytometry analysis and in vitro assay. These IgE⁺ plasmablasts readily secrete IgE, upregulate specific mRNA transcripts (BLIMP-1 IRF4, XBP1, CD138, and TACI), and exhibit highly differentiated morphology all consistent with plasmablast differentiation.

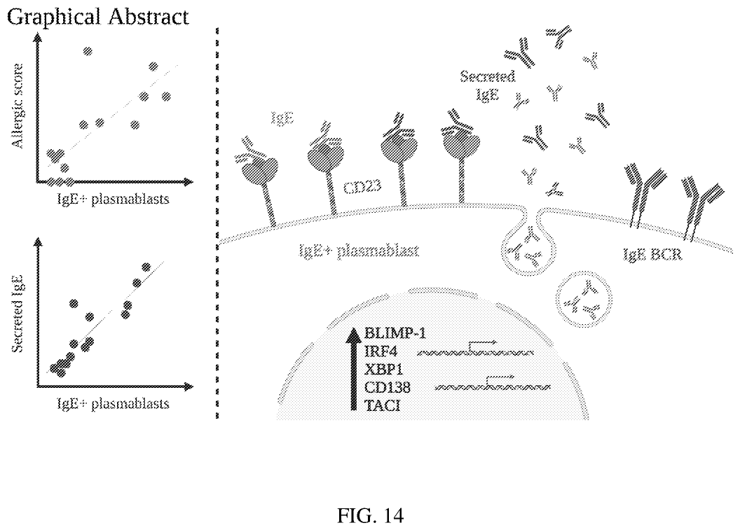

### DETAILED DESCRIPTION

One aspect of the current disclosure is directed to IgE+ plasmablasts as a diagnostic biomarker for monitoring individuals at risk for developing allergy, such as those with a genetic predisposition. In such an aspect, the biomarker can be measured to determine whether an allergy is developing, or not, in individuals at risk for developing allergy. For example, these could be children with one or both parents allergic. In some embodiments, the biomarker is measured twice, once when there is no allergen exposure to set a normal baseline for the individual, and once during allergen exposure to determine if the biomarker is elevated. For seasonal allergies, the first measurement occurs outside of the allergy season and the second measurement should during the allergy season. For food allergies, the subject fasts from potential allergens before the first measurement and the potential allergens are introduced before the second measurement.

Another aspect of the current disclosure is directed to IgE+ plasmablasts as a diagnostic biomarker as a treatment success indicator for allergen immunotherapy or other biological allergy treatments. Before beginning therapy, the biomarker is measured 1-2 times to create a normal baseline for the individual. If possible, measure the biomarker when the individual is experiencing allergic signs and when the individual is in allergy remission to establish the biomarker range and individual's biomarker profile. For example, the biomarker could then be measured approximately one week following each allergen injection to monitor whether the immunotherapy parameters are working effectively to desensitize the individual or not. Injections (allergen type and dose, injection site, frequency of injections) that decrease the biomarker can be established and continued to develop an optimized treatment regime for each patient. Injections that do not change the biomarker should be modified for the individual.

Another aspect of the current disclosure is directed to IgE+ plasmablasts as a diagnostic biomarker to confirm if clinical signs are caused by allergic reactions or other reasons. IgE+ plasmablasts are used as a diagnostic biomarker to diagnose if new clinical signs in a subject are due to an allergic reaction or not. In this case, the biomarker should be measured as soon as possible during, or immediately (e.g. 1-2 days) following clinical signs. If the biomarker value is above the set threshold for that species, the individual is at risk of being allergic and the clinician can proceed with further tests to diagnose the causal allergen(s). If the biomarker is below the set threshold, a second timepoint can be measured one month later for confirmation and other non-allergy causes should be investigated.

The disclosed addresses three problems that have currently no reliable solution: (1) There are no reliable diagnostic biomarkers to predict the development and progression of allergic diseases. It is not yet possible to determine if an individual is developing allergy before clinical signs become visible. At the time clinical allergy is apparent, the patient's immune system has already dysregulated various immune functions that are not easy to reverse once started. (2) In addition, there is no diagnostic biomarker to monitor the effectiveness of allergen immunotherapy. Allergen immunotherapy is the only current approved allergy treatment that is not just symptomatic but tries to cure and reverse the immune dysregulation leading to allergy. A predictive and diagnostic allergy biomarker can be used to determine if the dosage, timing and site of injections and/or allergen selection is working to desensitize and heal the individual. As a result, allergen immunotherapy is often long-lasting, unsuccessful and expensive. (3) Finally, there are no rapid and repeatable methods to identify and study IgE-secreting cells in the context of allergy, thereby limiting the ability to use these cells as diagnostics. All currently available methods to identify and/or analyze IgE-secreting cells require a complex marker panel which is complicated and lacks repeatability in clinical diagnostic applications.

Currently, no technologies address these three problems. The disclosure herein improves upon several aspects of existing technologies. First, in humans, soluble allergen-specific IgE in serum is used as a diagnostic marker. However, allergen-specific IgE concentrations are low in serum and can vary due to the short half-life of IgE in the circulation. This makes the threshold of detection problematic to define. Both false negative and false positive reactions often occur by using tests that detect allergen-specific IgE in serum. The use of IgE as a biomarker also depends on identification of the specific allergens that an individual is allergic to and is often not consistent in all individuals. In horses and other veterinary species, major allergens are not always defined. Thus, allergen-specific IgE detection is challenging due to the lack of available reagents and/or low concentration in serum. Total serum IgE has sometimes been evaluated instead and is not a consistent biomarker. This invention circumvents these challenges by using the cellular source of IgE (IgE+ plasmablasts) as the biomarker, instead of the product (soluble serum IgE).

Second, IgE+ plasmablasts have been identified in humans, however the experimental approach to identify IgE+ plasmablasts is complex, difficult to analyze, and challenging to repeat. Various research groups have worked on this task and have not been able to clearly identify and distinguish IgE secreting cells from IgG secreting cells. This is due to CD23-bound IgE on the surface of IgE+ plasmablasts. IgE+ plasmablasts, unlike other plasmablasts, uniquely express CD23, previously preventing the distinction from other B cells, which all also express CD23. Therefore, difficult approaches, often based on multiple cell surface markers, had to be designed to address this problem. The disclosed method circumvents this challenge by using a novel acid wash approach to remove CD23-bound IgE and enable simple and rapid quantification of IgE+ plasmablasts in peripheral blood.

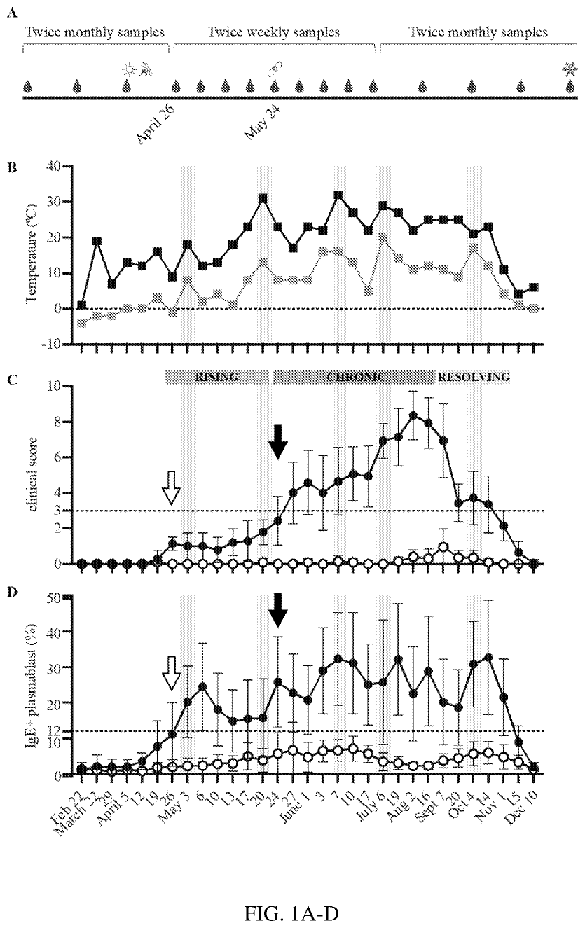

The disclosure provides a solution to each of these problems by identifying a novel predictive biomarker that 1) is detectable before clinical allergy, 2) is dynamic and responds quickly to environmental changes, such as allergen exposure and treatment, and 3) can be rapidly and quantitatively measured with the disclosed acid wash method (FIG. 1).

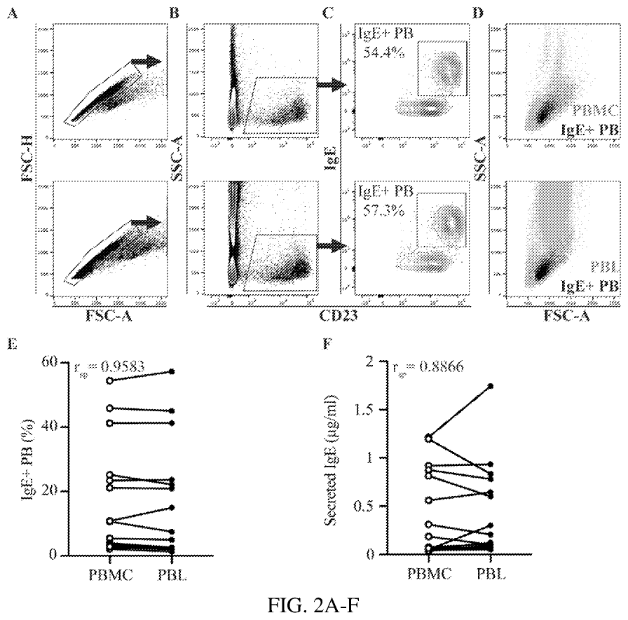

In one aspect, the disclosure is directed to a method of detecting IgE+ plasmablasts in a mammal. In some embodiments, the methods comprise a first step (i) of contacting peripheral blood leukocytes (PBL) or peripheral blood mononuclear cells (PBMC) isolated from a blood sample of the mammal, with an acidic wash solution, and thereafter collecting the cells. In some embodiments, the methods comprise a second step (ii) of contacting the collected cells from step (i) with a detection agent directed to IgE. In some embodiments, the methods comprise a third step (iii) of detecting IgE+ cells as IgE+ plasmablasts (FIG. 2).

As used herein, “plasmablasts” are plasma cell precursors which undergo cell division, unlike fully differentiated plasma cells. Plasmablasts produce various immunoglobulin isotypes. One such isotype is IgE. Immunoglobulin E (IgE) is the antibody that causes allergic reactions through binding of effector cells and stimulation by specific allergens. IgE is produced when allergen-specific B cells are activated by allergen in the presence of the cytokines IL-4 and IL-13, leading to class switch recombination of the constant region to express the IGHE gene. These class-switched B cells, now expressing an IgE B cell receptor, then receive additional survival signals and begin differentiating into memory B cells and plasma cells, which secrete high concentrations of (soluble) IgE into circulation. During this activation process, some IgE+ B cells differentiate into IgE+ plasmablasts. IgE+ plasmablasts secrete antibody and enter peripheral blood, providing a snapshot of the IgE+ B cell to plasma cell differentiation process simultaneously occurring in the lymph node or local tissue. Peripheral IgE+ plasmablasts may continue to the bone marrow where they further differentiate into plasma cells. Plasmablasts are characterized by both Ig surface expression and secretion. As used herein, “IgE+ plasmablasts” are cells which express IgE on the cell surface and secrete IgE. IgE+ plasmablasts can be found in peripheral blood.

CD23, a C-type lectin expressed on the surface of activated B cells in mammals, is a low affinity IgE receptor. CD23 has different roles in B cell signaling and is most often assumed to decrease IgE production and subsequent allergy. IgE binding to CD23 decreases the availability of IgE for sensitization on mast cells and basophils, and also downregulates IgE production. However, CD23 on B cells is also associated with the facilitation of antigen presentation. During antigen presentation, allergen/IgE complexes are internalized and presented on B cell MHCII molecules, or transferred to dendritic cells, to promote epitope spreading and T cell activation, thereby promoting allergic responses. While CD23 is expressed on most activated peripheral B cells, recent single cell sequencing of B cell surface proteins identified that some human individuals have two distinct CD23+ B cell populations (CD23^{lo} and CD23^{hi}). This suggests heterogeneity in CD23 function on B cells. However, the cause and clinical relevance of these two populations was not explored. Similarly, another recent study noticed that allergic humans have CD23^{hi} class switched B cells that are absent in healthy controls. However, due to CD23-bound IgE, this unique CD23^{hi} B cell population was not further characterized. In addition, increased CD23 surface density on B cells has been positively correlated with allergen-specific IgE levels in allergic individuals, further supporting that variations in B cell CD23 expression may occur in the context of allergy. This suggests that CD23^{hi} cells may have clinical relevance during allergic diseases. As used herein, “CD23+ B cells” refers to B cells expressing CD23 on the cell surface.

As used herein, “peripheral blood leukocytes” or “PBL” are a group of closely related cells, including neutrophils, monocytes, eosinophils, basophils, and lymphocytes. These cells continuously move throughout the body by means of the blood stream, lymphatics, and their ability to migrate through tissues. In some instances, PBL are commonly known as white blood cells. As used herein, “peripheral blood mononuclear cells” or “PBMC” are identified as any blood cell with a round nucleus, i.e. lymphocytes, monocytes, natural killer cells (NK cells) or dendritic cells. PBMC are isolated from peripheral blood. Methods of isolating PBL and PBMC are known in the art. In some embodiments, methods of isolating PBL and PBMC occur through methods explained below. In some embodiments, the PBLs or PBMCs are obtained by removing red blood cells from a blood sample of the mammal. In humans, this can be done by a short hypotonic lysis step or by using an erythrocyte separator tube. In horses, heparinized peripheral blood can be left in collection tubes for approximately 30 minutes at room temperature to allow the erythrocytes to settle and leukocyte-rich plasma (peripheral blood leukocytes, PBL) can be collected. In some embodiments, samples do not need to be processed in a sterile biosafety cabinet.

In some embodiments, the acidic wash solution has a pH of 3.1 or lower. In some embodiments, the acidic wash solution has a pH range of pH 2.5-3.1. In some embodiments, the acidic wash solution has a pH in the range of pH 2.8-3.0. In some embodiments, the acidic wash solution has pH of about pH 3.0 or less. In some embodiments, the acidic wash solution is any acidic solution having a pH of about 3.1 or less. In some embodiments, the acidic wash solution has pH of about pH 2.9 or less. In some embodiments, the acidic wash solution is any acidic solution having a pH of about 2.8 or less. In some embodiments, the acidic wash solution has pH of about pH 2.7 or less. In some embodiments, the acidic wash solution is any acidic solution having a pH of about 2.6 or less. In some embodiments, the acidic wash solution has pH of about pH 2.5 or less. In some embodiments, the acidic wash solution is a lactic acid. In some embodiments, the acidic wash solution is a citric acid. In some embodiments, the acidic wash solution is a hydrochloric acid. In some embodiments, the acidic wash solution may be a combination of acids.

In some embodiments, the isolated PBL or PBMC are contacted with the acidic wash solution for about 3-10 minutes. In some embodiments, the isolated PBL or PBMC are contacted with the acidic wash solution for about 10 minutes. In some embodiments, the isolated PBL or PBMC are contacted with the acidic wash solution for about 9 minutes. In some embodiments, the isolated PBL or PBMC are contacted with the acidic wash solution for about 8 minutes. In some embodiments, the isolated PBL or PBMC are contacted with the acidic wash solution for about 7 minutes. In some embodiments, the isolated PBL or PBMC are contacted with the acidic wash solution for about 6 minutes. In some embodiments, the isolated PBL or PBMC are contacted with the acidic wash solution for about 5 minutes. In some embodiments, the isolated PBL or PBMC are contacted with the acidic wash solution for about 4 minutes. In some embodiments, the isolated PBL or PBMC are contacted with the acidic wash solution for about 3 minutes.

In some embodiments, the isolated PBL or PBMC are washed with a wash solution prior to the step of contacting them with an acidic wash solution. In some embodiments, the wash solution is not a buffered solution. In some embodiments, the wash solution is at a pH below 7.0. In some embodiments, the wash solution is pH 6.8 or lower. In some embodiments, the wash solution is pH 6.6 or lower. In some embodiments, the wash solution is pH 6.4 or lower. In some embodiments, the wash solution is pH 6.2 or lower. In some embodiments, the wash solution is pH 6.1 or lower. In some embodiments, the wash solution is pH 6.0 or lower. In some embodiments, the wash solution is pH 5.9 or lower. In some embodiments, the wash solution is pH 5.8 or lower. In some embodiments, the wash solution is pH 5.7 or lower. In some embodiments, the wash solution is pH 5.6 or lower. In some embodiments, the wash solution is pH 5.5 or lower. In some embodiments, the wash solution is pH 5.4 or lower. In some embodiments, the wash solution is pH 5.2 or lower. In some embodiments, the wash solution is pH 5.0 or lower.

In some embodiments, steps (ii) and (iii) comprise (ii) contacting the collected cells from step (i) with a detection agent directed to IgE; and (iii) detecting IgE+ cells as IgE+ plasmablasts. In some embodiments, the detection agent directed to IgE is a monoclonal antibody. In some embodiments, the detection of IgE+ cells is achieved through flow cytometry.

In some embodiments, steps (ii) and (iii) comprise (ii) contacting the collected cells from step (i) with a detection agent directed to CD23 and a detection agent directed to IgE; and (iii) detecting CD23+ and IgE+ cells as IgE+ plasmablasts. In some embodiments, the detection agent directed to CD23 is a monoclonal antibody. In some embodiments, the detection agent directed to IgE is a monoclonal antibody. In some embodiments, the detection of CD23+ and IgE+ cells is achieved through flow cytometry.

In some embodiments, different fluorochromes are conjugated to the two monoclonal antibodies to fit the available flow cytometer. Fluorochromes that are excited by two different lasers, if possible, can be used to eliminate the need for compensation. Alexa fluorochromes were used herein due to the simplicity of the manufacturer's protocol for antibody-fluorochrome conjugation, and the slow bleach rate of the dye. However, other fluorochromes can be used.

In some embodiments, the PBL or PMBC are then incubated for flow cytometry in PBS-BSA with two monoclonal antibodies (anti-IgE, e.g., conjugated to Alexa fluorochrome 647, and anti-CD23, e.g., conjugated to Alexa fluorochrome 488). Cells are incubated for 15 minutes at room temperature in the dark and then washed once with 1 ml PBS-BSA. Samples are recorded on a flow cytometer. To analyze, flow cytometry data image is gated first to exclude doublets (FSC-A x FSC-H plot, gate selects cells along the diagonal) and second to gate on CD23+ cells (gate selects cells in the right half of a plot showing CD23 on the x axis and SSC-A on the y axis). A third gate is applied to cells in the upper right quadrant of a CD23 (x axis) x IgE (y axis) plot (FIG. 2 shows an example of this gating strategy). These are IgE+ plasmablasts and the percentage of IgE+ plasmablasts out of CD23+ cells is the biomarker readout.

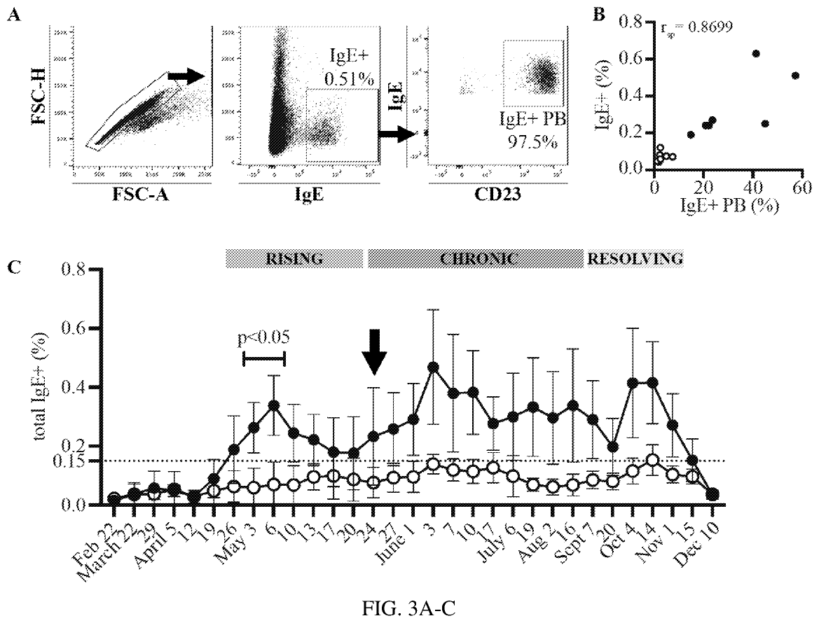

In some embodiments, cells are incubated with only an anti-IgE monoclonal antibody. In some embodiments, the monoclonal antibody is fluorescently conjugated. In some embodiments, analysis first excludes doublets (FSC-A x FSC-H plot, gate on cells along the diagonal) and second gate on IgE+ cells (gate selects cells in the right half of a plot showing IgE on the x axis and SSC-A on the y axis). The percentage of IgE+ out of total doublet-excluded events would be the diagnostic readout (FIG. 3).

In some embodiments, the pH is neutralized after the PBL or PBMC are contacted with the acidic wash solution but before collecting the cells. In some embodiments, the pH is neutralized after the acidic wash solution by using Tris-HCl buffer solution at pH7-8. In some embodiments, the pH is neutralized with PBS. In some embodiments, the pH is neutralized with another similar buffered solution with a neutral pH.

As used herein, “mammal” is any mammal. In some embodiments, the mammal is a human, horse, dog, or cat. As used herein, “subject” is any mammal from which the blood sample has been obtained.

In some embodiments, the percentage of IgE+ cells in the total CD23+ cells is determined.

Some embodiments of the disclosure are directed to methods of quantifying IgE+ plasmablasts, the methods comprising culturing peripheral blood leukocytes (PBL), or peripheral blood mononuclear cells (PBMC) isolated from a blood sample of a mammal, measuring IgE secreted from the cultured cells in the supernatant; and correlating the level of secreted IgE with the level of IgE+ plasmablasts. In some embodiments, the secreted IgE is measured by a laminar flow device, ELISA, bead-based assays, or similar assay platforms known in the art. In some embodiments, the level of IgE+ plasmablasts reflects the percentage of IgE+ cells in the total CD23+ cells.

In some embodiments, the PBL or PBMC are cultured for a period of about 6 hours to about 96 hours. In some embodiments, the PBL or PBMC are cultured for a period of about 12 hours to about 90 hours. In some embodiments, the PBL or PBMC are cultured for a period of about 18 hours to about 84 hours. In some embodiments, the PBL or PBMC are cultured for a period of about 24 hours to about 72 hours.

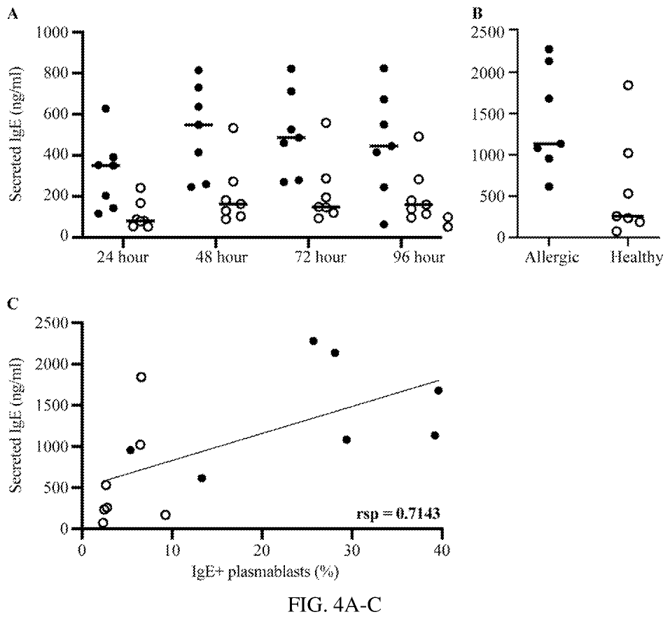

In some embodiments, secreted IgE from IgE+ plasmablasts is measured as a quantitative readout. In some embodiments, the disclosure is directed to methods of quantifying secreted IgE comprising culturing PBL or PBMC isolated from a blood sample of a mammal, measuring IgE secreted from the cultured cells in the supernatant, and quantifying the level of secreted IgE. In some embodiments, secreted IgE directly correlates to the frequency of IgE+ plasmablasts in peripheral blood (see FIG. 4). In some embodiments, secreted IgE can be measured by Luminex bead-based assay, ELISA, rapid lateral flow detection tests, or any other chemiluminescent protein detection assay, or similar assay platforms known in the art.

In some embodiments, an ELISpot assay is used to measure the frequency of IgE-secreting plasmablasts in a subject as a quantitative readout. In some embodiments, plates are coated with allergen or anti-IgE antibodies, then PBL or whole blood samples added to the plate and incubated. This allows all IgE+ plasmablasts to secrete IgE, which binds to the anti-IgE antibody (or allergen). Secreted IgE could then be quantified in spots to determine the frequency of IgE+ plasmablasts.

In some embodiments, the disclosed acid wash approach is rapid, providing a turn-around time of 2 hours from retrieval of the sample to quantitative result. In some embodiments, the methods can be performed in a non-sterile environment. In some embodiments, the methods require minimal flow cytometer capacity or user technical background.

A further aspect of the disclosure is directed to a method of predicting clinical allergy in a mammal, the method comprising detecting IgE+ plasmablasts in the mammal, and determining the risk of developing clinical allergy based on the level of IgE+ plasmablasts. In some embodiments, the IgE+ plasmablasts are detected using the methods disclosed herein. In some embodiments, the level of IgE+ plasmablasts is determined based on the percentage of CD23+ and IgE+ cells in the total CD23+ cells. In some embodiments, the level of IgE+ plasmablasts is determined based on the percentage of IgE+ cells in the total sample. In some embodiments, the disclosure is directed to a method of determining the severity of clinical allergy in a mammal, comprising detecting IgE+ plasmablasts in the mammal, and determining the severity of clinical allergy based on the level of IgE+ plasmablasts.

In some embodiments, the mammal will likely develop clinical allergy if the percentage of IgE+ cells in the total CD23+ cells is above 10%. In some embodiments, the mammal will likely develop clinical allergy if the percentage of IgE+ cells in the total CD23+ cells is above 11%. In some embodiments, the mammal will likely develop clinical allergy if the percentage of IgE+ cells in the total CD23+ cells is above 12%. In some embodiments, the mammal will likely develop clinical allergy if the percentage of IgE+ cells in the total CD23+ cells is above 13%. In some embodiments, the mammal will likely develop clinical allergy if the percentage of IgE+ cells in the total CD23+ cells is above 14.%.

In some embodiments, the mammal will likely develop clinical allergy if the percentage of IgE+ cells in the total cells is above 0.1%. In some embodiments, the mammal will likely develop clinical allergy if the percentage of IgE+ cells in the total cells is above 0.11%. In some embodiments, the mammal will likely develop clinical allergy if the percentage of IgE+ cells in the total cells is above 0.12%. In some embodiments, the mammal will likely develop clinical allergy if the percentage of IgE+ cells in the total cells is above 0.13%. In some embodiments, the mammal will likely develop clinical allergy if the percentage of IgE+ cells in the total cells is above 0.14%. In some embodiments, the mammal will likely develop clinical allergy if the percentage of IgE+ cells in the total cells is above 0.15%.

In some embodiments, the method further comprises treating the mammal with antihistamines, allergen immunotherapy, or biologics such as monoclonal antibodies or cell-based therapies aimed to desensitize the individual to allergens when it is determined that the mammal will likely develop clinical allergy.

In some embodiments, the method further comprises treating the mammal by dialyzing the IgE+ CD23+ cells isolated from the mammal. In some embodiments, the mammal is a human, a horse, a dog, or a cat. In some embodiments, the dialysis runs through a low pH chamber to remove surface-bound IgE, followed by a chamber with an anti-IgE monoclonal antibody. A threshold to remove cells fluoresce due to that antibody would be set to selectively remove IgE+ cells. The acid wash chamber would prevent accidental removal of normal CD23+ B cells. In some embodiments, subjects with autoimmune disease undergo dialysis to remove IgE+ cells.

Another aspect of the disclosure is directed to a method of predicting clinical allergy in a mammal, the method comprising detecting secreted IgE in the mammal, and determining the risk of developing clinical allergy based on the level of secreted IgE. In some embodiments, the secreted IgE is detected using the methods disclosed herein. In some embodiments, the disclosure is directed to a method of determining the severity of clinical allergy in a mammal, comprising detecting secreted IgE in the mammal, and determining the severity of clinical allergy based on the level of secreted IgE.

In some embodiments, the mammal will likely develop clinical allergy if the level of secreted IgE passes a predetermined threshold. In some embodiments, the predetermined threshold is a detected level of secreted IgE above 200 ng/mL. In some embodiments, the predetermined threshold is a detected level of secreted IgE above 300 ng/mL. In some embodiments, the predetermined threshold is a detected level of secreted IgE above 400 ng/mL. In some embodiments, the predetermined threshold is a detected level of secreted IgE above 500 ng/mL. In some embodiments, the predetermined threshold is a detected level of secreted IgE above 600 ng/mL. In some embodiments, the predetermined threshold is a detected level of secreted IgE above 700 ng/mL. In some embodiments, the predetermined threshold is a detected level of secreted IgE above 800 ng/mL. In some embodiments, the predetermined threshold is a detected level of secreted IgE above 900 ng/mL. In some embodiments, the predetermined threshold is a detected level of secreted IgE above 1,000 ng/mL. In some embodiments, the predetermined threshold is a detected level of secreted IgE above 1,500 ng/mL.

In some embodiments, the method further comprises treating the mammal with antihistamines, allergen immunotherapy, or biologics such as monoclonal antibodies or cell-based therapies aimed to desensitize the individual to allergens when it is determined that the mammal will likely develop clinical allergy.

Another aspect of the disclosure is directed to a method for monitoring progression of an allergy comprising monitoring the level of IgE+ plasmablasts in a mammal, and determining that allergy is progressing based on an increase in the level of IgE+ plasmablasts. In some embodiments, the IgE+ plasmablasts are detected using methods disclosed herein. In some embodiments, the level of IgE+ plasmablasts is determined based on the percentage IgE+ cells in the total CD23+ cells. In some embodiments, the level of IgE+ plasmablasts is determined based on the percentage of IgE+ cells in the total sample. In some embodiments, the mammal is a human, a horse, a dog, or a cat.

In some embodiments, the progression of an allergy is based on monitoring the efficacy of a treatment regimen for the allergy, comprising monitoring the level of IgE+ plasmablasts in a mammal undergoing the treatment regimen for allergy, and determining the efficacy of the treatment regimen based on the level of IgE+ plasmablasts. In some embodiments, a decrease in the level of IgE+ plasmablasts indicates that the treatment regimen is effective. In some embodiments, the IgE+ plasmablasts are detected using methods disclosed herein. In some embodiments, the level of IgE+ plasmablasts is determined based on the percentage of CD23+ and IgE+ cells in the total CD23+ cells. In some embodiments, the level of IgE+ plasmablasts is determined based on the percentage of IgE+ cells in the total sample. In some embodiments, the mammal is a human, a horse, a dog, or a cat.

Another aspect of the disclosure is directed to a method for monitoring progression of an allergy comprising monitoring the level of secreted IgE in a mammal, and determining that allergy is progressing based on an increase in the level of secreted IgE. In some embodiments, the secreted IgE is detected using methods disclosed herein. In some embodiments, the mammal is a human, a horse, a dog, or a cat.

In some embodiments, the progression of an allergy is based on monitoring the efficacy of a treatment regimen for the allergy, comprising monitoring the level of secreted IgE in a mammal undergoing the treatment regimen for allergy, and determining the efficacy of the treatment regimen based on the level of secreted IgE. In some embodiments, a decrease in the level of secreted IgE indicates that the treatment regimen is effective. In some embodiments, the secreted IgE is detected using methods disclosed herein. In some embodiments, the mammal is a human, a horse, a dog, or a cat.

Another aspect of the disclosure is directed to a kit for detecting IgE+ plasmablasts, comprising an acidic wash solution and a monoclonal anti-IgE antibody. In some embodiments, the kit further comprises instructions for use. In some embodiments, the kit further comprises a monoclonal anti-CD23 antibody. In some embodiments, the anti-CD23 antibody and the anti-IgE antibody are of different species. In some embodiments, the anti-CD23 antibody is against horse CD23 and the anti-IgE antibody is against horse IgE. In some embodiments, the anti-CD23 antibody is against human CD23 and the anti-IgE antibody is against human IgE. In some embodiments, the kit further comprises labeled detection antibodies against the anti-CD23 antibody and the anti-IgE antibody monoclonal. In some embodiments, the monoclonal anti-CD23 antibody and the monoclonal anti-IgE antibody are coupled to different color fluorescent beads.

In some embodiments, the kit is for clinical use. In some embodiments, the kit is for research purposes.

Ultimately, the methods described herein provide clear quantitative results that allow for objective interpretation.

Some embodiments of the disclosure are directed to using smaller volumes of leukocyte-rich plasma and still ensuring accurate quantification of IgE+ plasmablasts. In some embodiments, at least 500 μl of leukocyte-rich plasma is used. In some embodiments, at least 400 μl of leukocyte-rich plasma is used. In some embodiments, at least 300 μl of leukocyte-rich plasma is used.

Some embodiments of the disclosure are directed to using smaller volumes of whole blood and still ensuring accurate quantification of IgE+ plasmablasts. In some embodiments, at least 500 μl of whole blood is used. In some embodiments, at least 400 μl of whole blood is used. In some embodiments, at least 300 μl of whole blood is used.

In some embodiments of the disclosure, heparinized blood samples are stored at 4° C. for 1-4 days, or longer, before processing. In some embodiments, when the heparinized blood samples are ready to be processed, blood tubes are gently inverted multiple times before processing the sample. In some embodiments, 1-2 ml of leukocyte-rich plasma is collected and used with the acid wash of the disclosure. In some embodiments, 1-2 ml of whole blood is collected and used with the acid wash protocol of the disclosure.

In some embodiments, erythrocytes are removed from the sample. In humans, this can be done by a short hypotonic lysis step or by using an erythrocyte separator tube. In horses, heparinized peripheral blood can be left in collection tubes for approximately 30 minutes at room temperature to allow the erythrocytes to settle and leukocyte-rich plasma (peripheral blood leukocytes, PBL) can be collected. In some embodiments, samples do not need to be processed in a sterile biosafety cabinet. 1 ml PBL is collected and pelleted at 500×g 10 min at 4° C. All subsequent wash steps are performed at 100 g for 5 min at 4° C. Cells are washed twice with 1 ml ice-cold wash solution (130 mM NaCl, 5 mM KCl; pH 6). Then, cells are resuspended in ice-cold acid wash solution (10 mM lactic acid (Alfa Aesar, Thermo Fisher Scientific, Lancashire, UK), 130 mM NaCl, 5 mM KCl; pH 2.8-3) and incubated for 5 min on ice. After incubation, 0.2 ml of 1 M Tris HCl, pH 8 (Thermo Fisher Scientific, Waltham, MA, USA) is added to neutralize the acid before pelleting the cells by centrifugation. Cells are washed once with 1 ml PBS and fixed in 2% (v/v) paraformaldehyde solution (PFA, Sigma-Aldrich, St. Louis, MO, USA) for 20 minutes at room temperature, then washed twice in 1 ml PBS and once in 1 ml PBS-BSA (0.5% (w/v) BSA, 0.02% (w/v) NaN3, all from Sigma-Aldrich, St. Louis, MO, USA). Wash steps after formalin fixation are performed at room temperature.

In some embodiments, the formalin-fixation step is skipped, and live cells are incubated with antibodies and analyzed. In some such embodiments, steps of the disclosed acid wash methods are performed on ice. In some embodiments, steps of the disclosed acid wash methods are performed on the same day as sample collection. In some embodiments, steps of the disclosed acid wash methods are performed on ice and on the same day as sample collection.

Some embodiments of the disclosure are directed to an allergy treatment. In some embodiments, allergic individuals following allergen exposure undergo a dialysis-like treatment where their peripheral blood B-cells are acid washed and labeled for IgE+ cells. The IgE+ plasmablasts are removed from circulation and the rest of the blood cells are immediately buffered to become isotonic, and replaced into the subject. Such embodiments could decrease the progression of the allergic response through the removal of the source of IgE, interrupting the vicious cycle of IgE production during allergic diseases.

Some embodiments of the disclosure are directed to diagnostic tests measuring secreted IgE. In some embodiments, a rapid secreted IgE assay kit is used for allergy doctors to measure as a correlate for IgE+ plasmablasts. In some embodiments, this occurs in the doctor's office. In some embodiments, a peripheral blood sample is collected and added to an incubation well for an incubation at 37° C. Following incubation, the culture supernatant is harvested and added to a rapid lateral flow device to measure secreted IgE. Secreted IgE correlates directly with the frequency of the IgE+ plasmablast biomarker providing a simple, quick, and reliable quantification method. In some embodiments, the measuring occurs in an outsourced laboratory. In some embodiments, the measuring occurs in a local laboratory. In some embodiments, the measuring occurs in the lab of a doctor's office. In some embodiments, the measuring occurs in the doctor's office.

Some embodiments of the disclosure are directed to research test kits. In some embodiments, the acid wash approach to clearly identify and isolate IgE+ plasmablasts will allow scientific research advancement to study their roles during the pathomechanisms of allergy. In some embodiments, the kit comprises pre-made solutions. In some embodiments, live cells can be stained with the described antibodies and sorted by flow cytometry for functional and/or transcriptomic analysis. In some embodiments, sorted IgE+ plasmablasts are immortalized to generate purified allergen-specific monoclonal IgE. In some embodiments, the immortalized IgE+ plasmablasts are cloned. In some embodiments, the immortalized IgE+ plasmablasts are used to study the mechanisms of IgE+ plasmablasts (i.e. allergen binding and internalization, downstream signaling pathways, etc.).

Some embodiments of the disclosure are directed to allergen-specific diagnostics and allergen discovery. In some embodiments, IgE+ plasmablasts from a subject are purified and used to determine the specific allergen panel the subject is sensitized to. In such embodiments, the allergen-specificity of IgE secreted by IgE+ plasmablasts is measured. Such embodiments may have higher sensitivity than serum IgE measurement.

CD23-bound IgE on B cells and plasmablasts exist as a heterotrimer in complex with allergen. Therefore, some embodiments comprise using the disclosed acid wash for the removal of isolation of IgE/allergen complexes. In some embodiments, purified allergen from the isolated IgE/allergen complexes are characterized by mass spectroscopy to identify candidate allergens.

In some embodiments, the disclosure is directed to autologous IgE+ plasmablast therapy. In some embodiments, isolated IgE+ plasmablasts are used to model an allergic immune response to optimize and guide allergen immunotherapy in a research setting. In some embodiments, autologous IgE+ plasmablasts are harvested from a subject and frozen during clinical allergy. Then the autologous IgE+ plasmablasts are injected to the subject during remission, in the absence of naturally occurring allergens. The immune response following allergen injections during allergy remission is studied. In some embodiments, the injection of autologous IgE+ plasmablasts in the absence of allergen induces a regulatory and desensitization pathway, contributing to a form of immunotherapy.

### EXAMPLES

The following examples are presented to illustrate the present disclosure. The examples are not intended to be limiting in any manner.

### Example 1. Isolation of Peripheral Blood Leukocytes (PBL) and Peripheral Blood Mononuclear Cells (PBMC) and Acid Wash Removal of Receptor-Bound IgE

Heparinized blood samples settled at room temperature for at least 30 minutes to allow erythrocytes to separate from the cell-rich plasma. Cell rich plasma (1 ml), containing peripheral blood leukocytes (PBL), was collected and centrifuged at 500×g for 10 minutes at 4° C. PBL were then treated with an acid wash. A total of 3×10⁶ PBL were washed in 1 ml ice-cold wash solution (130 mM NaCl, 5 mM KCl; pH 6). Cells were then resuspended in ice-cold acid wash solution (10 mM lactic acid (Alfa Aesar, Thermo Fisher Scientific, Lancashire, UK), 130 mM NaCl, 5 mM KCl; pH 2.8-3) and incubated for 5 min on ice. After incubation, 0.2 ml of 1 M Tris HCl, pH 8 (Thermo Fisher Scientific, Waltham, MA, USA) was added to neutralize the acid before pelleting the cells by centrifugation. All wash steps were performed at 150 g for 5 min at 4° C. Cells were washed once with 1 ml PBS and either used immediately for live cell sorting or fixed in 2% (v/v) paraformaldehyde solution (PFA, Sigma-Aldrich, St. Louis, MO, USA) for 20 minutes at room temperature. An aliquot of PBL without acid washing was fixed in 2% PFA as a control.

All centrifugations were performed at 100×g for 5 minutes at 4° C. Briefly, PBL were washed twice in ice-cold wash solution (130 mM NaCl, 5 mM KCl; pH 6). Cells were then resuspended in ice-cold acid wash solution (10 mM lactic acid (Alfa Aesar, Thermo Fisher Scientific, Lancashire, UK), 130 mM NaCl, 5 mM KCl; pH 2.8-3) and incubated for 5 min on ice. After incubation, 0.2 ml of 1 M Tris HCl, pH 8 (Thermo Fisher Scientific, Waltham, MA, USA) was added to neutralize the acid before pelleting the cells by centrifugation. Cells were washed once in phosphate buffered saline (PBS, Fisher Scientific, Waltham, MA, USA) and then fixed in 2% (v/v) paraformaldehyde solution (PFA, Sigma-Aldrich, St. Louis, MO, USA) for 20 minutes at room temperature.

In addition, peripheral blood mononuclear cells (PBMC) were isolated by layering of cell-rich plasma over Ficoll (Ficoll Plaque Plus, GE Healthcare, Chicago, IL, USA) at a 2:1 (v:v) ratio. Density gradient centrifugation and PBMC isolation was performed. Heparinized peripheral blood was left in collection tubes for approximately 1 hour at room temperature to allow the erythrocytes to settle. The leukocyte-rich plasma was layered on Ficoll (Ficoll Plaque Plus, GE Healthcare, Chicago, IL, USA) at a 2:1 (v:v) ratio, followed by centrifugation at 2000 g for 20 min at 10° C. without brakes. The interphase containing PBMC was collected into a 50 ml conical tube, prefilled with phosphate buffered saline (PBS, Fisher Scientific, Waltham, MA, USA). The pellet was recovered by centrifugation at 500 g for 15 min at room temperature and suspended in PBS. Cells were washed once more in PBS to remove platelets and recovered by centrifugation at 150 g for 5 min at room temperature. All subsequent centrifugations were carried out at 150 g for 5 min at 4° C. or room temperature.

A total of 3×10⁶ PBMC were washed in 1 ml ice-cold wash solution (130 mM NaCl, 5 mM KCl; pH 6). Cells were then resuspended in ice-cold acid wash solution (10 mM lactic acid (Alfa Aesar, Thermo Fisher Scientific, Lancashire, UK), 130 mM NaCl, 5 mM KCl; pH 2.8-3) and incubated for 5 min on ice. After incubation, 0.2 ml of 1 M Tris HCl, pH 8 (Thermo Fisher Scientific, Waltham, MA, USA) was added to neutralize the acid before pelleting the cells by centrifugation. All wash steps were performed at 150 g for 5 min at 4° C. Cells were washed once with 1 ml PBS and either used immediately for live cell sorting or fixed in 2% (v/v) paraformaldehyde solution (PFA, Sigma-Aldrich, St. Louis, MO, USA) for 20 minutes at room temperature. An aliquot of PBMC without acid washing was fixed in 2% PFA as a control.

### Example 2. In Vitro Cell Culture and Quantification of Secreted IgE

Following the same PBL isolation as described above, 1 ml cell-rich plasma was collected and centrifuged at 500×g for 10 minutes at 4° C. The cell-depleted plasma was collected and stored at −20° C. until analyzed. PBL were resuspended in cell culture medium (DMEM supplemented with 1% (v/v) non-essential amino acids, 2 mM L-glutamine, 50 μM 2-mercaptoethanol, 50 μg/ml gentamicin, 100 U/ml penicillin, 100 μg/ml streptomycin (Thermo Fisher Scientific, Waltham, MA, USA) and 10% FCS (Atlanta biological, Flowery Branch, GA, USA)), and then centrifuged at 100×g for 5 minutes at room temperature. PBL were resuspended to 1 ml in cell culture medium, and 100 μl of PBL from each individual was mixed with cell culture medium to a final volume of 200 μl and incubated in 96-well flat-bottomed plates (Corning Incorporated, Corning, NY, USA) at 37° C., 5% CO₂. After 72-hours, cell-free supernatants were collected and stored at 4° C. until IgE secretion analysis. All steps were performed in a sterile biosafety cabinet.

PBMC were also isolated as described and 5×10⁵ PBMC were plated per well in 200 μl cell culture medium. Cells were incubated as described above for PBL and supernatants were collected for IgE analysis. In some instances, isolated PBMC (6×10⁵ cells/well, containing an average calculated 5.2×10²-3.5×10³ IgE⁺ plasmablasts), sorted CD23^{hi}/IgE⁺ (IgE⁺ plasmablasts) and CD23^{lo}/IgE⁻ (IgE− B cells) cells (3×10³ sorted cells/well) were incubated without stimulation in cell culture medium. Cells were incubated in 96-well flat-bottom plates (Corning Incorporated, Corning, NY, USA) at 37° C., 5% CO₂ for 72-hours before cell-free supernatants were collected and stored at 4° C. until analysis.

Secreted equine IgE was measured in undiluted supernatants using a fluorescent bead-based Luminex assay. The assay was set up as previously described with a standard curve of six five-fold dilutions (3.2 ng/ml-10 μg/ml IgE) (doi: published 26). Twelve 1:2 serial dilutions of purified IgE were used as a standard with concentrations ranging from 4.9 ng/ml-10 μg/ml total IgE. Total IgE was also measured in the cell-depleted plasma using eight 1:2 serial dilutions of purified IgE with concentrations ranging from 78.1 ng/ml-10 μg/ml total IgE, allowing measurement of the higher concentration of IgE in serum.

### Example 3. Equine CD23⁺ B Cells Include IgE Class Switched Cells

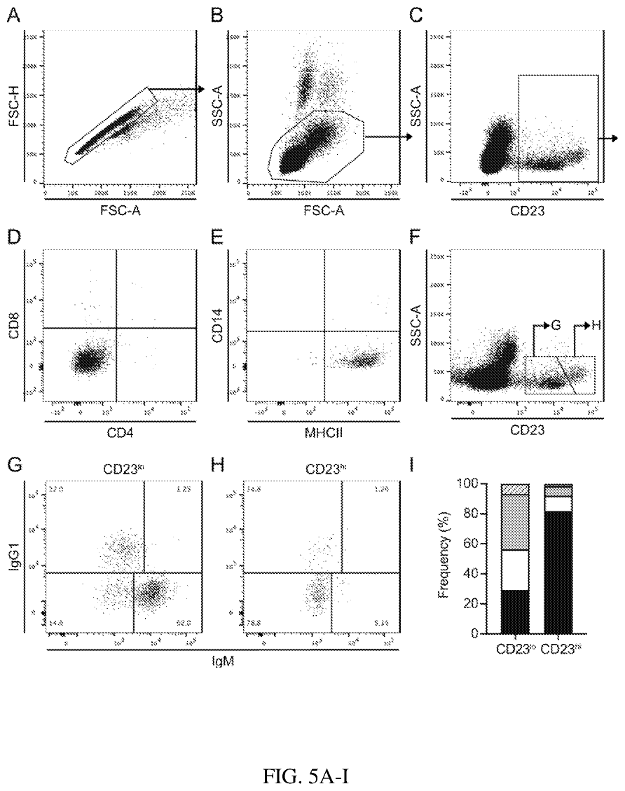

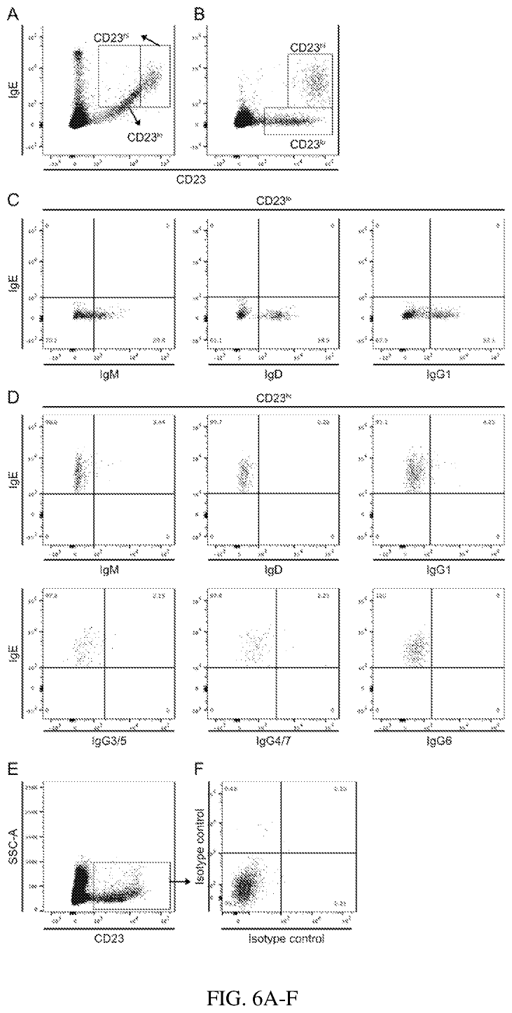

PBMC were stained with different mAbs and analyzed by flow cytometry to further characterize the CD23+ cell population in peripheral blood. CD23+ cells lacked T cell (CD4 and CD8) and monocyte (CD14) surface proteins but expressed high levels of MHCII (FIG. 5A-E). In allergic horses with clinical allergy, two distinct subpopulations could be separated with low and high CD23 expression (FIG. 5F). While the frequency of CD23^{lo} and CD23^{hi} subpopulations varied at different timepoints, the distribution of immunoglobulin isotypes expressed in each CD23 subpopulation was similar. CD23^{lo} cells were comprised of 7.0% IgM⁺/IgG1⁺ cells, 36.7% IgM⁺ cells, 27.0% IgG1⁺ cells, and 29.4% IgM⁻/IgG1⁻ cells (FIG. 5G, I, average of 4 individuals). In contrast, CD23^{hi} cells were comprised of 1.4% IgM⁺/IgG1⁺ cells, 6.7% IgM⁺ cells, 10.0% IgG1⁺ cells, and 81.9% IgM⁻/IgG1⁻ (FIG. 5H, I, average of 4 individuals). Circulating CD23+ cells were also IgE⁺ (FIG. 6A). Then, an approach to further characterize the CD23^{hi}/IgM⁻/IgG1⁻ cell population was developed.

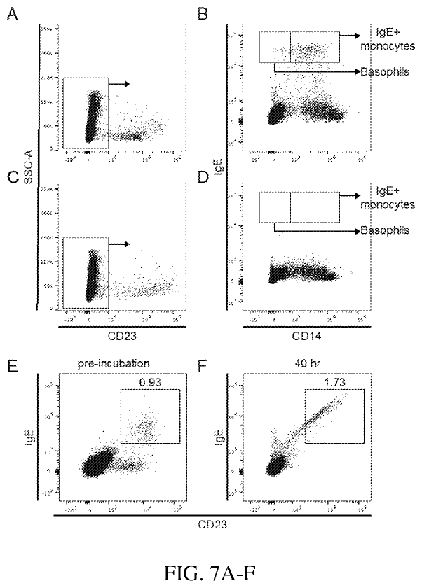

It was assumed that many of the CD23⁺ cells had bound IgE from circulation to their cell surface via the low affinity IgE-receptor CD23. Most of the IgE could be removed from CD23+ cells by a short lactic acid wash leaving two populations, CD23^{lo}/IgE⁻ and CD23^{hi}/IgE⁺ (FIG. 3B) cells. In addition, the lactic acid wash removed IgE from FcRI⁺ cells such as CD23⁻/CD14⁺ monocytes and CD23⁻/CD14⁻ basophils (FIG. 7A-D). After the acid wash, CD23^{lo} cells lost all surface IgE, but retained IgM, IgD or IgG1 (FIG. 6C) on the cell surface. This suggested that these cells were B cells expressing an IgM, IgD or IgG1 B cell receptor (BCR). From here forward, the CD23^{lo} population is referred to as IgE⁻ B cells (IgE− BC). In contrast, CD23^{hi} cells were still IgE⁺ after acid wash. The majority of CD23^{hi} cells did not express any other isotype and were IgM⁻ (99.7%), IgD⁻ (98.0%), IgG1⁻ (95.1%), IgG3/5⁻ (98.3%), IgG4/7-(98.9%), and IgG6⁻ (99.0%) (FIG. 6D, average of 4 allergic individuals). This demonstrated that these cells do not express any other isotype except IgE. Acid washed cells were labeled with anti-CD23 mAb (FIG. 7E) and also stained with mouse IgG1 isotype controls (FIG. 6F). Together, these data support that CD23^{hi}/IgE+ cells are IgE expressing B cells with an IgE BCR on their cell surface.

### Example 4. Peripheral IgE+ Plasmablasts are Rapidly Identified in Peripheral Blood Leukocytes (PBL)

IgE+ plasmablasts were quantified in peripheral blood by a short lactic acid wash (pH 3), followed by incubation with fluorescent antibodies against IgE and CD23, and quantification by flow cytometry as shown in Example 2. To quantify the IgE+ plasmablast frequency more rapidly in many individuals, this approach was adapted from isolated PBMC (FIG. 2A-D top images) to PBL (FIG. 2A-D bottom images). PBL isolation only required erythrocyte removal or lysis. In both sample types, a similar gating strategy was used where doublets were excluded (FIG. 2A), followed by gating on CD23+ cells (FIG. 2B), and then on IgE+ cells (FIG. 2C). IgE+ plasmablast identity was also confirmed by a larger forward (FSC) and side scatter (SSC), which is characteristic of these cells located between lymphocytes and monocytes in both PBMC and PBL (FIG. 2D). The frequency of IgE+ plasmablasts out of total CD23+ cells was similar between PBMC and PBL samples in all samples (FIG. 2E, n=17, rsp=0.9583, p<0.0001). Spontaneous secretion of IgE by in vitro culture of PBMC or PBL for 72 hours also resulted in similar concentrations of IgE in the supernatants (FIG. 2F, n=17, rsp=0.8866, p<0.0001).

### Example 5. CD23hi/IgE+ Cells Produce Intracellular IgE

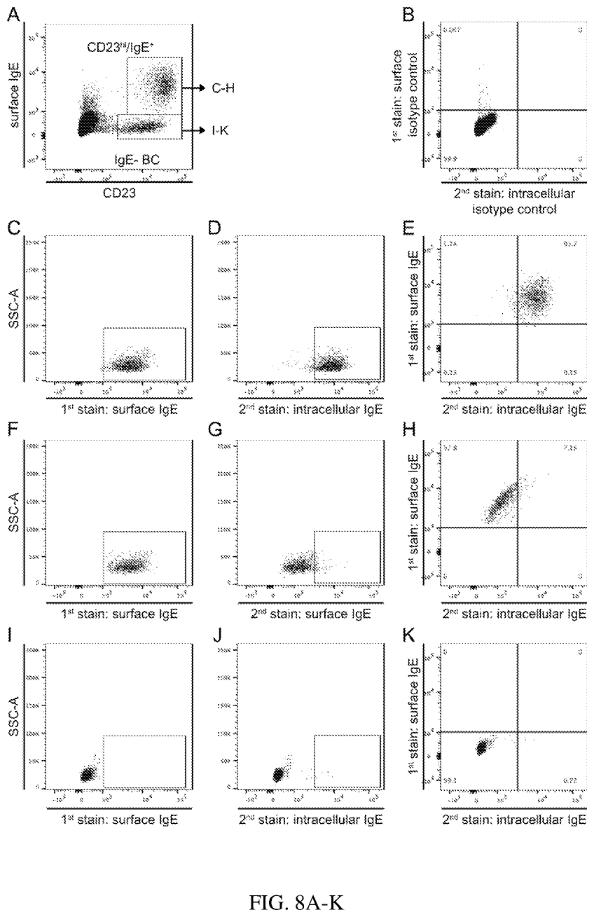

To further confirm if CD23hi/IgE+ cells produce IgE, CD23-bound IgE was again removed from PBMC by acid wash (FIG. 8A). Acid washed cells did not bind isotype controls extracellularly or intracellularly (FIG. 8B). The cells were stained with subsequent antibody master mixes to label them first for cell surface IgE (IgE mAb 176 coupled to Alexa Fluor 647, AF647), followed by intracellular IgE staining (IgE mAb 176 coupled to Alexa Fluor 488, AF488). The majority of CD23hi/IgE+ cells (92.9%, average of 4 allergic horses) expressed both cell surface (FIG. 8C, E) and intracellular IgE (FIG. 8D, E). A small fraction (6.8%, average of 4 allergic horses) of CD23hi/IgE+ cells expressed surface IgE only and no intracellular IgE. Less than 0.1% of CD23hi/IgE+ cells expressed intracellular IgE with no surface IgE.

The surface labeling of IgE saturated all surface IgE mAb binding sites and therefore intracellular staining revealed only intracellular IgE. This was confirmed by two sequential extracellular IgE staining steps. The majority of the CD23hi/IgE+ cells were only positive for the first IgE label (FIG. 8F, H) and the second labeling step did not label any additional surface IgE (FIG. 8G, H). Finally, IgE− B cells were negative for all surface (FIG. 8I, K) and intracellular IgE labeling (FIG. 8J, K). Together, these data further confirm that CD23hi/IgE+ cells have undergone class switching to IgE and consequently express IgE intracellularly and as part of their cell surface BCR.

### Example 6. CD23hi/IgE+ Cells Express Plasmablast and Plasma Cell Differentiation Genes

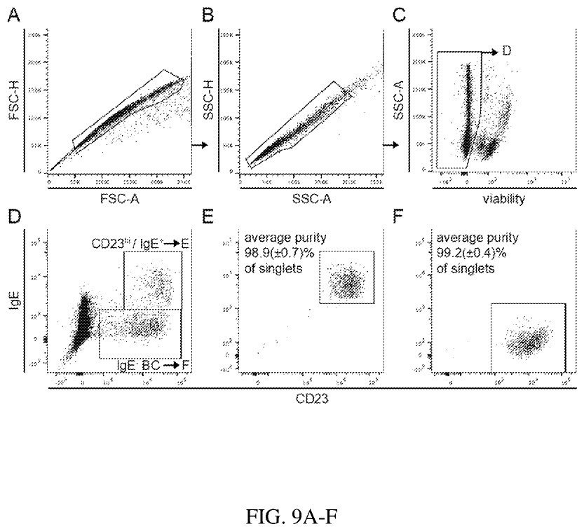

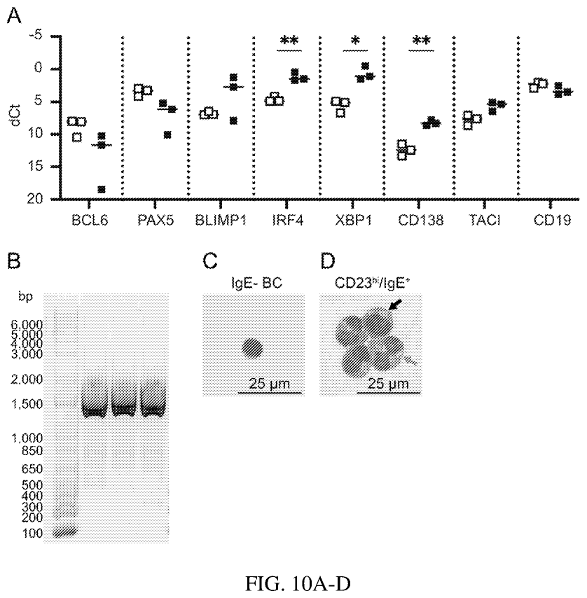

Before becoming plasma cells, B cells differentiate into plasmablasts which secrete antibody and travel from the lymph node through peripheral blood (doi: 10.3389/fimmu.2012.00078; doi: 10.1016/B978-0-12-397933-9.00014-X). To find additional evidence for the differentiation stage of peripheral CD23hi/IgE+ cells as antibody secreting cells, we used the RNA extracted from FACS sorted CD23hi/IgE+ cells and RNA extracted from IgE− B cells sorted simultaneously as controls (FIG. 9). Gene expression for B cell, plasmablast, and plasma cell differentiation was determined with gene specific primers (Table 1) by quantitative RT-PCR. Each sample was normalized to beta actin (ACTB, FIG. 10A) and normalized gene expression (dCt) was compared between CD23hi/IgE+ cells and IgE− B cells. Normalization with each reference gene resulted in similar trends in gene expression.

Plasmablasts and plasma cells downregulate transcription factor paired box protein 5 (PAX5) and B cell lymphoma 6 (BCL6) and upregulate B lymphocyte-induced maturation protein 1 (BLIMP-1), interferon regulating factor 4 (IRF4) and x-box binding protein 1 (XBP1) (Table 1). An expression pattern was detected similar to this while comparing the mean difference of expression (ddCt) in CD23hi/IgE+ cells and IgE− B cells. Positive ddCt values represent increased expression in CD23hi/IgE+ cells and vis versa. CD23hi/IgE+ cells had decreased expression of PAX5 (ddCt=−3.67, p=0.144) and BCL6 (ddCt=−4.63, p=0.118) compared to IgE− B cells (FIG. 10A). In contrast, CD23hi/IgE+ cells had increased expression of BLIMP-1 (ddCt=2.833, p=0.322), IRF4 (ddCt=3.417, p=0.002), and XBP1 (ddCt=4.877, p=0.012) compared to IgE− B cells (FIG. 10A). CD23hi/IgE+ cells also had increased expression of transcripts for surface proteins CD138 (ddCt=4.177, p=0.005) and transactivator and CAML interactor (TACI, ddCt=2.117, p=2.117), compared to IgE− B cells (FIG. 10A). CD138 and TACI both begin to be expressed as B cells differentiate to plasmablasts and plasma cells. CD23hi/IgE+ cells had similar expression of B cell marker CD19 compared to IgE− B cells (ddCt=−0.907, p=0.057, FIG. 10A). Overall, these gene expression profile trends further support peripheral CD23hi/IgE+ cells as plasmablasts compared to IgE− B cells.

To verify that CD23hi/IgE+ cells had undergone class switching and were truly expressing IgE, expression of the IgE heavy chain constant region (IGHE) RNA transcript was measured by PCR using IGHE-specific primers (Table 1). The PCR product from CD23hi/IgE+ cell cDNA was 1272 base pairs (FIG. 10B) and was sequenced and confirmed to be the expressed IGHE cDNA sequence with 99% homology to the annotated sequence (Table 1). Therefore, this sequence was referred to as the equine IGHEc haplotype. This provides yet another proof that these cells have undergone class switching and express IgE.

Additionally, morphology of CD23hi/IgE+ cells and IgE− B cells were explored by differential staining of sorted cell populations. Consistent with B cell morphology, IgE− B cells had characteristic round, central nuclei with coarsely clumped chromatin, a narrow cytoplasmic area and small cell size (<10 μm diameter; FIG. 10C). In contrast, CD23hi/IgE+ cells were much larger (>10 μm diameter; FIG. 10D) with central nuclei and coarse chromatin. The CD23hi/IgE+ cells also had perinuclear clear zone (FIG. 10D, black arrow), a larger and deeply basophilic cytoplasm, and occasional small clear vacuoles, (FIG. 10D, gray arrow). This is indicative of an expanded golgi apparatus, higher cytoplasmic protein content and secretory vacuoles, respectively, and is consistent with antibody secreting cells. In summary, CD23hi/IgE+ cells show typical gene expression and morphology characteristics of peripheral IgE+ plasmablasts. Throughout this disclosure, CD23hi/IgE+ cells are referred to as IgE+ plasmablasts (IgE+ PB).

TABLE 1
Primer pairs to differentiate memory B cells, plasmablasts and plasma cells in IgE+ peripheral
blood cells.

| | | | | | | | SEQ |
|---|---|---|---|---|---|---|---|
| Gene | Gene | Lineage Stageª | | | Accession | | ID |
| type | Name | MBC | PB | PC | number | Forward and Reverse primer | NO: |
| Ref gene | ACTB | + | + | + | XM_ | F: CCAGCACGATGAAGATCAAG | 1 |
| | | | | | 023655002 | R: GTGGACAATGAGGCCAGAAT | 2 |
| | B2M | + | + | + | NM_ | F: TCTTTCAGCAAGGACTGGTCTTT | 3 |
| | | | | | 001082502.3 | R: CATCCACACCATTGGGAGTAAA | 4 |
| TFs | BCL6 | + | − | − | XM_ | F: ATTCCAGCTGTGAGAATGGG | 5 |
| | | | | | 003363354.4 | R: CGGTCACACTTGTAGGGTTT | 6 |
| | PAX5 | + | − | − | JQ044379.1 | F: ACGCGTGTGTGACAATGA | 7 |
| | | | | | | R: CACTATGCTGTGGCTGGAA | 8 |
| | BLIMP-1 | − | + | ++ | XM_ | F: AGGAGCTTCTTGTGTGGTATTG | 9 |
| | | | | | 001501824.5 | R: CTTAGGATGGCTCTGTGTTTGT | 10 |
| | IRF4 | − | ++ | ++ | XM_ | F: CCCATAGAGCCAAGCATAAGG | 11 |
| | | | | | 023624331.1 | R: TTGACGTGGTCAGCTCTTTC | 12 |
| | XBP1 | − | ++ | ++ | XM_ | F: CTGGAACAGCAAGTGGTAGAT | 13 |
| | | | | | 014742035 | R: CAGGCGCTGTCTTAACTCTT | 14 |
| Cell | CD19 | + | lo/− | − | NM_ | F: AGTCCCTGCTGCAACTTTAG | 15 |
| surface | | | | | 001267799.2 | R: GGGATCAGTCATTCGCTTTCT | 16 |
| proteins | CD138 | − | −/+ | ++ | XM_ | F: TCCTGGACAGGAAGGAAGT | 17 |
| | | | | | 023619466.1 | R: GAGTAGCTGCCCTCATCTTTC | 18 |
| | TACI | − | − | + | XM_ | F: CCATCATCTGCTGCTTCCT | 19 |
| | | | | | 005598001.2 | R: GCTTCCATCCAGTGATCCTT | 20 |

| | | | | | |
|---|---|---|---|---|---|
| Ig | IGHE | Expressed in | AJ305047.1 | F: GTCTCCAAGCAAGCCCCATTA | 21 |
| | | IgE+ MBC, PB, | | R: TCGCAAGCTTTACCAGGGTCTT | 22 |
| | | and PC | | TGGACACCTC | |

### Example 7. Peripheral IgE+ Plasmablasts are Increased in Allergic Horses and Correlate with Clinical Allergy Severity

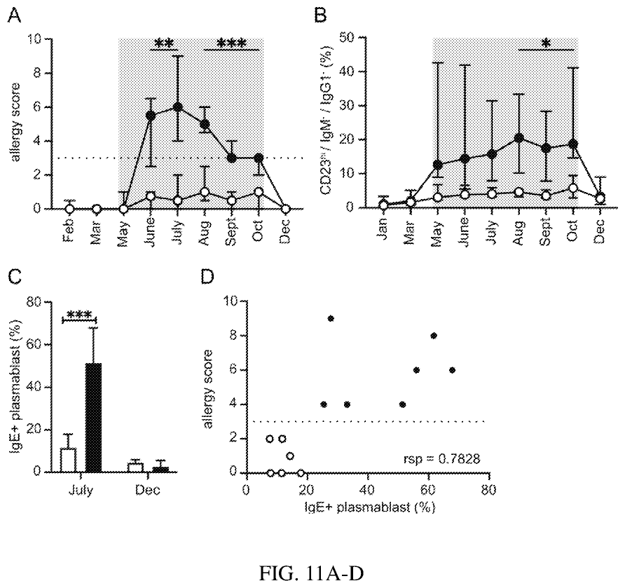

To determine whether IgE+ plasmablasts were elevated during clinical allergy, percentages were compared of IgE+ plasmablasts in the peripheral blood of healthy horses (n=6) with those of allergic horses with Culicoides (Cul) hypersensitivity (n=7). Cul hypersensitivity is a seasonal allergic disease and therefore allergic horses only develop clinical signs in the summer during Cul allergen exposure (Table 2, FIG. 11A). CD23^{hi} B cells were first measured in PBMC ex vivo, which still had CD23-bound IgE. As shown above, CD23hi/IgM−/IgG1− cells include, but do not entirely distinguish, IgE+ plasmablasts. The frequency of CD23hi/IgM−/IgG1− cells were compared between healthy and allergic horse groups for one year and it was determined that during the chronic phase of allergy, in late summer and in fall, allergic horses had significantly increased percentages of CD23hi/IgM−/IgG1− B cells compared to healthy controls (FIG. 11B).

TABLE 2
Clinical scores of Cul hypersensitivity of allergic and healthy
horses living together in the same environment.

| | | Allergy score, median (range) | |
|---|---|---|---|
| | Cul | Allergic horses | Healthy horses |
| Month | exposure | (n = 7) | (n = 6) |
| February | none | 0 (0-0.5) | 0 (0-0) |
| March | | 0 (0-0) | 0 (0-0) |
| April | | 0 (0-0) | 0 (0-0) |
| May | onset^{c} | 0 (0-1) | 0 (0-0) |
| June | constant^{d} | 5.5 (2.5-6.5) | 0.75 (0-1) |
| July | | 6 (4-9) | 0.5 (0-2) |
| August | | 5 (4.5-6) | 1 (0.5-2.5) |
| September | | 3 (3-4) | 0.5 (0-1) |
| October | decline^{e} | 3 (2-3) | 1 (0-1) |
| November | none | 1.5 (1-2) | 0.25 (0-0.5) |
| December | | 1 (0-1) | 0 (0-0) |

Bolded rows (May-October) denote months with Cul allergen exposure. There was no allergen exposure from February-April and November-December. Clinical allergy scores ≥3 are considered allergic.

However, acid wash treatment of PBMC to remove CD23-bound IgE allows more accurate gating and identification of IgE+ plasmablasts. IgE+ plasmablasts were compared in acid washed PBMC. Allergic horses had significantly increased frequencies of IgE+ plasmablasts compared to healthy horses in July, but not in December when all horses appeared clinically healthy again (FIG. 11C). In fact, IgE+ plasmablasts comprised approximately 50% (median 52.4%, range 26.1-68.9%) of total CD23+ cells in allergic horses in the summer. This was in clear contrast to healthy horses, where only about 10% of CD23+ cells were IgE+ plasmablasts (median 11.9%, range 7.6-18.2%). Clinical scores and IgE+ plasmablast percentages were highly correlated in July when Cul allergen exposure and clinical allergy peaked in allergic horses (rsp=0.7828, p<0.01; FIG. 11D), demonstrating that IgE+ plasmablasts are indicative of allergy severity in Cul hypersensitivity.

### Example 8. Peripheral IgE+ Plasmablasts Secrete IgE, which is Elevated in Allergic Horses

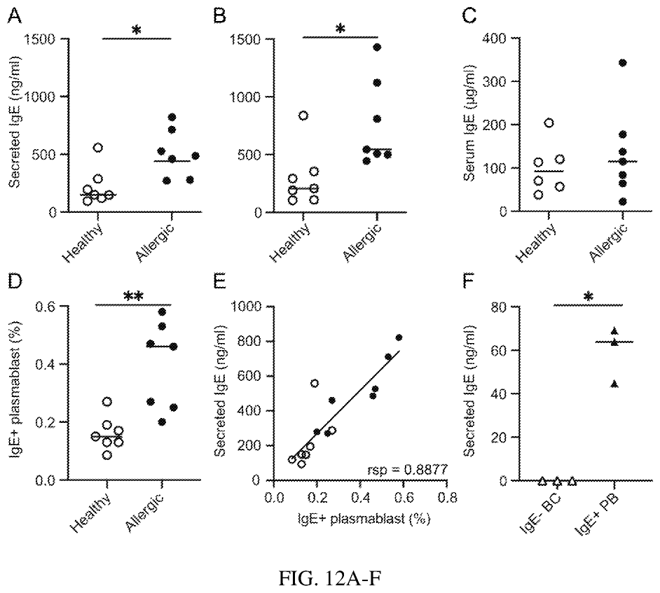

To determine if IgE+ plasmablasts are secreting IgE, isolated PBMC from allergic (n=7) and non-allergic horses (n=7) were cultured for 3 days in cell culture medium without additional stimuli. Afterwards, secreted IgE antibodies were measured in the supernatants. While PBMC from all horses secreted IgE, PBMC from allergic horses secreted significantly more IgE (median 486.2 ng/ml, range 270.8-822.0 ng/ml) than healthy horses (median 149.8 ng/ml, range 94.2-558.5 ng/ml) (FIG. 12A). PBMC were also treated with the acid wash solution to remove all CD23-bound IgE and subsequently cultured for 3 days. The concentrations of secreted IgE in the supernatant of these acid washed cells (FIG. 12B, median 544.8 ng/ml, range 444.7-1428.2 ng/ml in allergic horses) were similar compared to ex vivo incubated PBMC, and allergic horses had significantly higher secreted IgE concentrations compared to healthy controls. This difference in IgE secretion was not reflected in serum. Total IgE was measured in serum samples and was similar between allergic (median 114.7 μg/ml, range 22.2-343.0 μg/ml) and healthy horses (median 92.0 μg/ml, range 38.4-203.9 μg/ml) (FIG. 12C).

An aliquot of ex vivo PBMC from each horse was treated with the acid wash solution and analyzed by flow cytometry to compare the percentage of IgE+ plasmablasts between groups (FIG. 12D). The concentration of secreted IgE was proportional to the frequency IgE+ plasmablasts in total PBMC (FIG. 12E, rsp=0.8877, p<0.0001). IgE+ plasmablasts from allergic horses were also sorted (see FIG. 9) and incubated for 3 days without additional stimuli. Sorted IgE− B cells were included as a control. IgE+ plasmablasts, but not IgE− B cells, from allergic horses secreted IgE (FIG. 12F). In summary, these data indicate that IgE+ plasmablasts are actively secreting IgE, and that IgE secretion is enhanced in allergic compared to healthy horses due to the increased numbers of IgE+ plasmablasts in allergic individuals.

Secreted IgE by IgE+ plasmablasts likely first binds to available IgE receptors, and then contributes to soluble IgE in the cell culture supernatant. To test this, PBMC were acid washed (FIG. 7E) and then incubated for 40-hours. After incubation, all CD23+ cells re-bound secreted IgE through surface CD23 (FIG. 7F), demonstrating that CD23+ cells, including IgE+ plasmablasts, are functional after the short acid treatment and can bind secreted IgE.

### Example 9. IgE Secretion by IgE+ Plasmablasts Correlates with Disease Severity

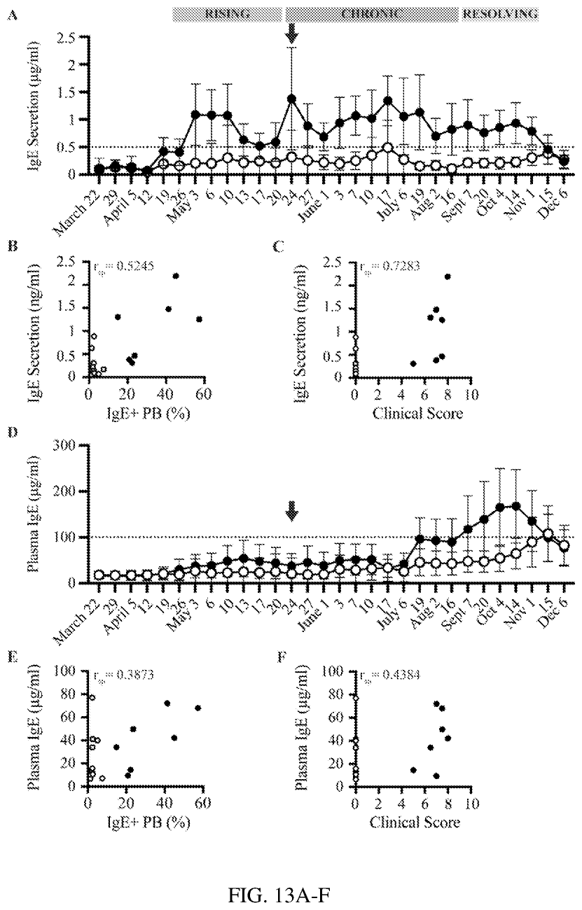

IgE+ plasmablasts spontaneously secrete IgE as shown herein. IgE secretion by these cells was measured after 72 hours of in vitro culture of PBL. The concentration of secreted IgE from allergic and healthy horses followed a similar trend as the frequency of IgE+ plasmablasts (FIG. 13A). IgE secretion increased in allergic horses at the beginning of the “Rising Phase” on April 26, and was maintained above a threshold of 0.5 μg/ml for the duration of the summer. In general, IgE secretion by PBL from healthy horses stayed below 0.5 μg/ml. However, occasionally a healthy horse had elevated IgE secretion. In these instances, the frequency of IgE+ plasmablasts were also elevated by flow cytometry. Overall, spontaneous IgE secretion from PBL correlated positively with IgE+ plasmablast frequencies (rsp=0.5245, p<0.05, FIG. 13B) and clinical allergy scores (rsp=0.7283, p<0.005, FIG. 7C). IgE secretion can thus serve as another method to quantify IgE+ plasmablasts and predict onset of allergy.

In comparison to the spontaneous IgE secretion from PBL, IgE concentrations in plasma from the same blood samples were measured in all horses. Total IgE in plasma was only elevated in some allergic individuals later in the summer, at the end of the “Chronic Phase” and beginning of the “Resolving Phase”. On July 17, 4 out of 7 allergic horses had plasma IgE concentrations above the plasma IgE threshold of 100 μg/ml. Plasma IgE in 2 of the allergic horses did not cross the threshold until the “Resolving Phase” in October, 5 months after clinical allergy onset. One horse never reached the plasma IgE threshold (FIG. 13D). Plasma IgE concentrations, therefore, had a weak correlation with IgE+ plasmablast frequencies (rsp=0.3873, p=0.1255, FIG. 7E) and clinical allergy scores (rsp=0.4384, p=0.0797, FIG. 7F). In summary, the increase of IgE concentrations in plasma is delayed by at least 8 weeks (2 months) compared to clinical allergy, does not distinguish allergic from healthy horses before or during the onset of allergy, and cannot be used to predict disease.

### General Methodologies.

### Animals, Clinical Allergy Scoring, and Blood Collection.

Heparinized blood samples were obtained from the V. jugularis using the BD Vacutainer system (Becton Dickinson, Franklin Lakes, NJ, USA). All animals studied were Icelandic horses living together in the same environment with natural exposure to Cul from mid-May to mid-October. Cul were absent from the environment of the horses for the remainder of the year. Vaccination and deworming were synchronized. All horses were annually vaccinated against rabies, tetanus, West Nile virus and Eastern and Western Encephalitis virus, as well as dewormed with moxidectin and praziquantel (Zoetis, Parsipanny, NJ, USA) once a year in December. All horses were on the same diet. They were kept full time on large pastures with run-in-sheds, free access to water, mineral salt blocks, and were grazing in the summer and fed grass hay in the winter. All horses studied had either naturally occurring Cul hypersensitivity or were clinically healthy. Allergic horses (n=7) included 5 mares (11-17 years, median 16 years), 1 gelding (age 9) and 1 stallion (age 8). Healthy horses (n=6) included 4 mares (7-9 years, median 8 years) and 2 geldings (age 8). For CD23+ cell phenotyping experiments, 3 allergic mares, 1 allergic gelding, 1 stallion, and 4 healthy mares were used. For FACS sorting and PCR analysis 2 allergic mares and 1 allergic gelding were used. For longitudinal analysis of ex vivo peripheral blood mononuclear cells (PBMC), all 7 allergic and 6 healthy horses were compared at each timepoint except for March, which included 7 allergic and 4 healthy horses.

All horses were given a clinical allergy score every 2-4 weeks for the duration of the study. Scores were assigned based on pruritis (0-3), alopecia (0-4), and dermatitis (0-3) and total scores ≥3 were considered allergic. All allergic horses displayed allergic signs with scores above 3 for at least one year before, and for the duration of this study (Table 2). Cul-specific hypersensitivity was further confirmed by intradermal skin testing with Cul whole body extract (WBE; Stallergenes Greer Inc., Cambridge, MA, USA) in comparison to saline and histamine controls. Allergic horses developed an immediate skin reaction to Cul WBE. Nonallergic horses never exhibited clinical allergy or scoring above 3 and did not react to intradermal Cul injections.

### Cell Staining and Flow Cytometry Analysis

For extracellular staining and analysis, PBMC or PBL were incubated for 15 min with different antibody master mixes (Table 3) in PBS-BSA (0.5% (w/v) BSA, 0.02% (w/v) NaN3, all from Sigma-Aldrich, St. Louis, MO, USA). Monoclonal antibodies (mAbs) against different horse cell surface markers or Ig isotypes were conjugated to Alexa fluorochrome 647, Alexa fluorochrome 488, or biotin according to manufacturer's protocols (Thermo Fisher Scientific, Waltham, MA, USA). Master mixes 1-10 (Table 3) were used to phenotype the different CD23⁺ populations in equine PBMC. Master mixes 11-15 were used to measure intracellular IgE production. Master mix 16 was used for cell sorting of CD23^{hi}/IgE⁺ cells and IgE⁻ B cells. Master mix 17 was used to measure IgE+ plasmablasts in allergic and healthy horses at different timepoints. Streptavidin-PE (Jackson ImmunoResearch Laboratories) was used to label biotinylated mAbs.

For intracellular staining and analysis, fixed PBMC were incubated first with master mix 11 (Table 3) in PBS-BSA to label cell surface IgE and CD23. Cells were subsequently incubated with streptavidin-PE to label biotinylated CD23. Cells were then washed once in saponin buffer (0.5% Saponin 0.5% BSA 0.02% NaN3 in PBS, all from Sigma-Aldrich, St. Louis, MO, USA), and incubated with master mix 12 in saponin buffer to label intracellular IgE. As a control, one aliquot of cells was incubated with master mix 13, in PBS-BSA instead of saponin buffer. Isotype controls were included in master mixes 14 and 15, which were subsequently stained on an additional PBMC aliquot to set the IgE⁺ gates.

Samples were recorded on a BD FACS Canto II flow cytometer and data analysis was performed with FlowJo version 10.4 (FlowJo, Ashland, OR, USA). A total of 100,000 events/sample were recorded for all master mixes except mixes 2 and 3, which recorded 50,000 events/sample. All flow cytometry images were gated first to exclude doublets and second to gate on PBMC by forward and side scatter characteristics. IgE+ cells were analyzed quantitatively.

TABLE 3
Antibody master mixes used for
flow cytometry analysis or FACS

| Master | Alexa | Alexa | Phyco- | Pacific | EC/ |
|---|---|---|---|---|---|
| Mix | Fluor 647 | Fluor 488 | erythrin^{j} | Blue | IC^{o} |
| 1 | CD4^{a} | CD23^{f} | CD8^{k} | N/A | EC |
| 2 | CD14^{b} | CD23^{f} | MHCII^{l} | N/A | EC |
| 3 | IgG1^{c} | CD23^{f} | IgM^{m} | N/A | EC |
| 4 | IgE^{d} | CD23^{f} | IgG1^{c} | N/A | EC |
| 5 | IgE^{d} | CD23^{f} | IgM^{m} | N/A | EC |
| 6 | IgD^{e} | IgE^{d} | CD23^{f} | N/A | EC |
| 7 | CD23^{f} | IgE^{d} | IgG3/5ⁿ | N/A | EC |
| 8 | IgG4/7^{g} | IgE^{d} | CD23^{f} | N/A | EC |
| 9 | IgG6^{h} | IgE^{d} | CD23^{f} | N/A | EC |
| 10 | Isotype | CD23^{f} | Isotype | N/A | EC |
| | controlⁱ | | controlⁱ | | |
| 11 | IgE^{d} | N/A | CD23f | N/A | EC |
| 12 | N/A | IgE^{d} | N/A | N/A | IC |
| 13 | N/A | IgE^{d} | N/A | N/A | EC |
| 14 | Isotype | N/A | CD23^{f} | N/A | EC |
| | controlⁱ | | | | |
| 15 | N/A | Isotype | N/A | N/A | IC |
| | | controlⁱ | | | |
| 16 | IgE^{d} | CD23^{f} | N/A | Live/ | EC |
| | | | | Dead | |
| 17 | IgE^{d} | CD23^{f} | CD14^{b} | N/A | EC |

### Fluorescence Activated Cell Sorting (FACS) of IgE+ Plasmablasts

Following PBMC isolation and removal of surface-bound IgE, 1×10⁷ cells were incubated with mAb master mix 16 (Table 3), including a viability marker (Thermo Fisher Scientific, Waltham, MA, USA), to isolate IgE⁺ plasmablasts by FACS. IgE mAb 176 was used for cell sorting due to its inability to induce crosslinking of receptor-bound IgE. All cell sorting was performed at 4° C. to minimize activation of B cells. Cells were sorted on a BD FACS Aria Fusion Sorter at the Cornell Institute of Biotechnology's flow cytometry core facility. Sorting was performed through a 100 μm nozzle at 20 psi in a sterile hood. Compensation was calculated with input from single stained UltraComp beads and amine reactive beads (Thermo Fisher Scientific, Waltham, MA, USA). Live CD23^{hi}/IgE⁺ cells were collected into cell culture medium (DMEM supplemented with 1% (v/v) non-essential amino acids, 2 mM L-glutamine, 50 μM 2-mercaptoethanol, 50 μg/ml gentamicin, 100 U/ml penicillin, 100 μg/ml streptomycin (Thermo Fisher Scientific, Waltham, MA, USA) and 10% FCS (Atlanta biological, Flowery Branch, GA, USA)) at 4° C. Live CD23^{lo}/IgE⁻ cells were simultaneously sorted and collected for comparison. Sorted fractions were counted and tested for viability using trypan blue exclusion. All samples were >88% viable (average 93% live cells). To confirm morphology of sorted fractions, cytospin smears were prepared in a cytocentrifuge (Thermo Shandon Cytospin 4, Thermo Fisher Scientific, Waltham, MA, USA) at 176 g for 4 min at room temperature, and then stained with Wright's stain. Morphology descriptions were performed by a single observer who was blinded to the origin of the cells and the sorting procedure.

### Primer Design and Amplification of B Cell and Plasmablast Differentiation Genes

Sorted CD23^{hi}/IgE⁺ cells and IgE⁻ B cells (1×10⁴ cells each) from three allergic horses in October were frozen in 3:1 Trizol LS:cell culture medium (Thermo Fisher Scientific, Waltham, MA, USA) at −80° C. until processed for RNA extraction. RNA was extracted in the aqueous layer with chloroform (Thermo Fisher Scientific, Waltham, MA, USA) in Phaselock Gel Heavy tubes (VWR International, Radnor, PA, USA). RNA pellets were co-precipitated with GlycoBlue (Ambion Incorporated, Austin, TX, USA) and washed once with isopropanol and three times with ethanol. Pellets were air dried and resuspended in 20 μl nuclease-free water (GrowCells, Irvine, CA, USA). RNA concentration and quality were measured via nanodrop. Equal amounts of extracted RNA were converted to cDNA using SuperScript™ VILO™ Reverse Transcriptase (RT) and no-Reverse Transcriptase control (no-RT) master mixes (Thermo Fisher Scientific, Waltham, MA, USA).

Primers specific for the equine IgE heavy chain constant region (IGHE) were synthesized by Integrated DNA Technology (Coralville, IA, USA). The transcript was amplified from 0.8 μl cDNA from FACS sorted CD23^{hi}/IgE⁺ cells, with 500 nM primers (Table 1, Forward primer: GTCTCCAAGCAAGCCCCATTA (SEQ ID NO: 21); reverse primer: TCGCAAGCTTTACCAGGGTCTTTGGACACCTC (SEQ ID NO: 22)) and Platinum™ Superfi PCR master mix (Invitrogen, Carlsbad, CA, USA). The PCR reaction was run with an initial denaturing of 94° C. for 10 seconds, then 30 cycles of 94° C. 10 sec, 57° C. 10 sec and 72° C. 30 sec, followed by a final elongation step at 72° C. 5 min and cooled to 4° C. PCR products were extracted from a 1% agarose gel (Invitrogen, Carlsbad, CA, USA), cloned into a pCR4-TOPO plasmid (Invitrogen, Carlsbad, CA, USA) and sequenced by Illumina Sequencing.

Additional gene specific primers (Table 1) were designed to span intron-exon boundaries with Integrated DNA Technology's PrimerQuest tool and were synthesized by Integrated DNA Technology (Coralville, IA, USA). Primers were also aligned with the equine genome and did not have any predicted off-target amplicons of similar product size. Therefore, to confirm primer specificity, PCR product size was confirmed on a 2% agarose gel (Invitrogen, Carlsbad, CA, USA) with a 10 bp ladder (Thermo Fisher Scientific, Waltham, MA, USA). Beta actin (ACTB) was used as a reference gene.

Quantitative real-time PCR (qPCR) was performed with 10 μl reactions each containing a cDNA equivalent obtained from a reverse transcriptase reaction of 3.4 ng mRNA, 500 nM forward and reverse primers and SsoAdvanced Universal SYBR green master mix (Bio-Rad Laboratories, Raleigh, NC, USA). No-RT controls were run simultaneously for all samples and primer pairs. The qPCR reaction was run on a QuantStudio 5 thermocycler (Thermo Fisher Scientific, Waltham, MA, USA) with an initial dissociation at 95° C. for 30 s, then 40 cycles of 95 C.° for 15 s and 60° C. for 15 s, followed by melt curve analysis. For qPCR results, delta Ct (dCt) values were calculated for all samples where dCt=Ct[target gene]−Ct[reference gene]. dCt values were calculated using both reference genes, ACTB or B2M. In general, the smaller the dCt, the greater the amount of mRNA in the sample. The mean difference in gene expression (ddCt) was calculated where ddCt is the average of dCt[IgE⁻ B cells]−dCt[CD23^{hi}/IgE⁺ cells] from each individual. Positive ddCt values represent increased gene expression in CD23^{hi}/IgE⁺ cells compared to IgE⁻ B cells.

### Statistical Analysis

D'Agostino and Pearson tests were performed on all data sets and confirmed that the data were not normally distributed. Thus, non-parametric tests were used for data analysis. To compare IgE⁺ plasmablast frequencies between allergic and healthy horses at different timepoints, a Holm-Sidak multiple comparisons test was used. A nonparametric Spearman rank correlation was calculated for all horses to compare each individual's clinical allergy score or concentration of secreted IgE to the peripheral IgE⁺ plasmablast percentage. A Mann-Whitney test was used to compare secreted IgE concentrations between allergic and healthy groups. A paired t test was used to compare dCt values between sorted CD23^{hi}/IgE⁺ cells and IgE⁻ B cells. Gene expression graphs plot individual and average dCt values for each gene and cell fraction. A paired t test was also used to compare IgE secretion between IgE⁻ B cells and IgE⁺ plasmablast FACS sorted samples. qPCR gene expression and IgE secretion were considered normally distributed due to the comparison of two cell types within each individual. All graphs plot median and range unless specified otherwise and p values <0.05 were considered significant. Analysis was performed with GraphPad Prism software version 8 (GraphPad Software Inc., La Jolla, CA, USA).

What is claimed:

1. A method of detecting IgE+ plasmablasts in a mammal, comprising:
(i) contacting peripheral blood leukocytes (PBL) or peripheral blood mononuclear cells (PBMC) isolated from a blood sample of the mammal, with an acidic wash solution, and thereafter collecting the cells;
(ii) contacting the collected cells from step (i) with a detection agent directed to IgE; and
(iii) detecting IgE+ cells as IgE+ plasmablasts.

2. The method of claim 1, comprising a wash step before step (i) wherein the wash step comprises contacting a sample with a wash solution wherein the wash solution has a pH in the range of pH 4.0-6.0.

3. The method of claim 1 or 2, wherein steps (ii) and (iii) comprise:
(ii) contacting the collected cells from step (i) with a detection agent directed to CD23 and a detection agent directed to IgE; and
(iii) detecting CD23+ and IgE+ cells as IgE+ plasmablasts

4. The method of any one of claims 1, 2, or 3, wherein the acidic wash solution has a pH in the range of 2.5-3.0, or 2.8-3.0.

5. The method of claim 4, wherein the cells are contacted with the acidic wash solution for about 3-10 minutes.

6. The method according to any one of claims 1-5, wherein the acidic wash solution is a lactic acid solution.

7. The method according to any one of claims 1-6, wherein the pH is neutralized before the cells are collected.

8. The method according to any one of claims 1-7, wherein the detection agent directed to CD23 is a monoclonal antibody, and the detection agent directed to IgE is a monoclonal antibody.

9. The method according to any one of claims 1-8, wherein the detection is achieved by a flow cytometer.

10. The method according to any one of claims 1-9, wherein the PBLs or PBMCs are obtained by removing red blood cells from a blood sample of the mammal.

11. The method according to any one of claims 1-10, wherein the mammal is a human, a horse, a dog, or a cat.

12. The method according to any one of claims 1-11, further determining the percentage of IgE+ cells in the total CD23+ cells.

13. A method of quantifying IgE, comprising
culturing peripheral blood leukocytes (PBLs) or peripheral blood mononuclear cells (PBMCs) isolated from a blood sample of the mammal,
measuring IgE secreted from the cultured cells in the supernatant; and
quantifying the level of secreted IgE.

14. The method of claim 13, wherein the culturing is performed for a period of about 6 hours to about 96 hours.

15. The method according to claim 13 or claim 14, wherein the quantification is achieved by a laminar flow device, ELISA, bead-based assays, or similar assay platforms.

16. The method according to claim 13 or claim 14, wherein the mammal is a human, a horse, a dog, or a cat.

17. The method according to any one of claims 13-16, wherein the level of secreted IgE reflects the percentages of IgE+ plasmablasts or IgE+ cells in the total CD23+ cells or IgE+ cells in total cells.

18. A method of predicting clinical allergy in a mammal, comprising
detecting IgE+ plasmablasts in the mammal or detecting secreted IgE from cultured PBLs or PMBCs of the mammal, and
determining the risk of developing clinical allergy based on the level of IgE+ plasmablasts or the level of secreted IgE.

19. A method of determining the severity of clinical allergy in a mammal, comprising
detecting IgE+ plasmablasts in the mammal or detecting secreted IgE from cultured PBLs or PMBCs of the mammal, and
determining the severity of clinical allergy based on the level of IgE+ plasmablasts or the level of secreted IgE.

20. The method of claim 18 or 19, wherein the IgE+ plasmablasts are detected using the method according to any one of claims 1-12, or wherein the secreted IgE is detected using the method according to any one of claims 13-17.

21. The method of claim 20, wherein the level of IgE+ plasmablasts is determined based on the percentage of IgE+ cells in the total CD23+ cells.

22. The method of claim 21, wherein the mammal will likely develop clinical allergy if the percentage of CD23+ and IgE+ cells in the total CD23+ cells is above a range of 10% to 14%.

23. The method according to claim 22, further comprising treating the mammal with antihistamines when it is determined that the mammal will likely develop clinical allergy.

24. The method according to claim 22, further comprising treating the mammal by dialyzing the IgE+ CD23+ cells isolated from the mammal.

25. The method according to any one of claims 18-24, wherein the mammal is a human, a horse, a dog, or a cat.

26. A method for monitoring progression of an allergy comprising:
monitoring the level of IgE+ plasmablasts in a mammal or the level of secreted IgE from cultured PBLs or PMBCs of the mammal, and
determining that allergy is progressing based on an increase in the level of IgE+ plasmablasts or the level of secreted IgE.

27. A method for monitoring the efficacy of a treatment regimen for allergy, comprising:
monitoring the level of IgE+ plasmablasts in a mammal or the level of secreted IgE from cultured PBLs or PMBCs of the mammal, wherein the mammal is undergoing the treatment regimen for allergy, and
determining the efficacy of the treatment regimen based on the level of IgE+ plasmablasts or the level of secreted IgE, wherein a decrease in the level of IgE+ plasmablasts or the level of secreted IgE indicates that the treatment regimen is effective.

28. The method of claim 26 or 27, wherein IgE+ plasmablasts are detected using the method according to any one of claims 1-12, or wherein the secreted IgE is detected using the method according to any one of claims 13-17.

29. The method according to any one of 26-28, wherein the level of IgE+ plasmablasts is determined based on the percentage of CD23+ and IgE+ cells in the total CD23+ cells; or
wherein the level of IgE+ plasmablasts is determined based on the percentage of IgE+ cells in the total cells.

30. The method according to any one of claims 26-29, wherein the mammal is a human, a horse, a dog, or a cat.

31. A kit for detecting IgE+ plasmablasts, comprising an acidic wash solution and a monoclonal anti-IgE antibody.

32. The kit of claim 31, further comprising an anti-CD23 antibody.

33. The kit of claim 31 or claim 32, further comprising instructions on how to use the kit.

34. The kit of any one of claims 31-33, wherein the anti-CD23 antibody and the anti-IgE antibody are of different species.

35. The kit of any one of claims 31-33, wherein the anti-CD23 antibody is against horse CD23 and the anti-IgE antibody is against horse IgE.

36. The kit of any one of claims 31-33, wherein the anti-CD23 antibody is against human CD23 and the anti-IgE antibody is against human IgE.

37. The kit of claim 31 or claim 33, wherein the monoclonal CD23 antibody and the monoclonal anti-IgE antibody are immobilized on a solid support.

38. The kit of claim 37, wherein the solid support is selected from the group consisting of a bead, a microwell plate, and a lateral flow device.

39. The kit of any one of claims 31-33, further comprising labeled detection antibodies against the anti-CD23 antibody and the anti-IgE antibody.

40. The kit of any one of claims 31-33, wherein the monoclonal anti-CD23 antibody and the monoclonal anti-IgE antibody are coupled to different color fluorescent labels.
